(19) United States
(12) Patent Application Publication
(10) Pub. No.: US 2021/0077478 A1
(43) Pub. Date: Mar. 18, 2021

(54) METHOD OF COUNTERING RESPIRATORY DEPRESSION VIA ACTIVATION OF NEURONAL HETEROMERIC NICOTINIC ACETYLCHOLINE RECEPTORS

(71) Applicant: THE GOVERNORS OF THE UNIVERSITY OF ALBERTA, Edmonton (CA)

(72) Inventors: JUN REN, Edmonton (CA); JOHN J. GREER, Edmonton (CA)

(21) Appl. No.: 16/766,429

(22) PCT Filed: Nov. 21, 2018
(86) PCT No.: PCT/CA2018/051482

§ 371 Date: May 22, 2020

Related U.S. Application Data
(60) Provisional application No. 62/589,834, filed on Nov. 22, 2017.
(60) Provisional application No. 62/611,305, filed on Dec. 28, 2017.

(51) Int. Cl. A61K 31/4439; A61K 31/4427; A61K 31/445; A61K 31/4406; A61K 31/40; A61K 31/422; A61K 31/55; A61P 23/00; A61P 11/16

(52) U.S. Cl. CPC A61K 31/4439; A61K 31/4427; A61K 31/445; A61K 31/4406; A61P 11/16; A61K 31/422; A61K 31/55; A61P 23/00; A61K 31/40

(57) ABSTRACT

Compounds capable of activating a neuronal heteromeric nicotinic acetylcholine receptor are provided and can be administered in the form of pharmaceutical compositions of the like. Methods for using the compounds for treating, preventing, or ameliorating respiratory depression and overdose induced by an opioid, or other cause of respiratory depression are also provided. Methods of inducing analgesia, anesthesia, or sedation in a subject, while simultaneously treating, preventing, or ameliorating respiratory depression and overdose induced by an opioid, or other cause of respiratory depression are also provided.

### FIELD OF THE INVENTION

The present invention relates to methods of using compounds which target neuronal heteromeric nicotinic acetylcholine receptors for treating, preventing, or ameliorating respiratory depression and overdose induced by an opioid, or other causes of respiratory depression in a subject including those caused by non-opioid drug, obstructive sleep apnea, central sleep apnea, apnea of prematurity, hypoxia, Prader-Willi Syndrome, Rett Syndrome, Pompe Disease, Cheyne-Stokes breathing, neuronal degeneration, stroke, heart failure, brain trauma, Parkinson's Disease, or spinal cord injury.

### BACKGROUND OF THE INVENTION

Opioid analgesics are the most widely used effective agents for treating acute, postoperative and chronic pain (Swarm et al., 2001). However, activation of opiate receptors leads to suppression of respiratory drive (Shook et al., 1990; Greer et al., 1995; Gray et al., 1999; Kivell et al., 2004; Lorier et al., 2010). A significant component of the reduction in respiratory frequency is due to binding of opioids to μ-opiate receptors expressed on inspiratory rhythm generating neurons within the preBötzinger complex (preBötC) (Gray et al., 2001; Montandon et al., 2011). Further, opioids depress hypoglossal (XII) motoneuron discharge and thus suppress the activation of the genioglossal muscle of the tongue that is important for maintaining an open airway (Lorier et al., 2010; Hajiha et al., 2009). Susceptibility to opioid-induced respiratory depression (OIRD) varies significantly among individuals (Desrosiers, 2006). Predicting which patients are most sensitive is difficult; however, older age, diseases affecting the cardiorespiratory system and sleep apnea are factors that raise patients' risk of harm from OIRD (Desrosiers, 2006; Agro et al., 2004; Launois et al., 2007). Patient controlled analgesia for extended periods is another area in which OIRD is a problem (i.e. hypoxemia or accidental death). The increase in the incidence of lethal overdose with use of oxycodone and fentanyl is also a major concern in North America and elsewhere (CCENDU Bulletin, 2015; CDC, 2017).

The current practice of administering the opioid receptor antagonist naloxone effectively counters OIRD but at the expense of losing analgesia. Thus, clinicians must find a balance of partial analgesia with manageable respiratory depression. Pre-clinical data indicate that activation of serotonin receptors can overcome OIRD (Guenther et al., 2009, 2010, 2012). Efficacy of a serotonergic receptor agonist against OIRD in a rat model has been demonstrated but at the expense of inducing marked side-effects (Ren et al., 2015). The pre-clinical data were consistent with clinical trials assessing serotonergic receptor agents against OIRD that have all shown a lack of efficacy at doses below those that cause serious side-effects (Lotsch et al., 2005; Oertel et al., 2007). Another strategy to counter OIRD has been to pharmacologically block BK_{Ca}-channels on carotid bodies and CNS neurons, such that hyperventilation is induced in rats and humans that partially balances out hypoventilation associated with OIRD (Golder et al., 2013; McLeod et al., 2014; Roozekrans et al., 2015). However, the research program appears to have ceased and was not developed sufficiently to determine clinical applicability (http://www.galleonpharma.com).

Drugs known as ampakines, which positively modulate AMPA (amino-3-hydroxy-5-methyl-4-isoxazolepropionic acid) receptors, were found to counter OIRD in rodent models (Gray et al., 1999; Ren et al., 2006; 2009). Similarly, data from a Phase IIa trial showed efficacy of an ampakine in humans exposed to modest levels of respiratory depression induced by alfentanil (Oertel et al., 2010). However, an ampakine was ineffective in a second Phase Ha clinical trial using an administration of remifentanil bolus that caused rapid and profound respiratory depression (Krystal et al., 2017). More potent ampakines may be more effective but there are concerns regarding excitation of the CNS (Lynch, 2006; Lynch and Gall, 2006). Solubility limitations of ampakines result in only oral formulations being currently available for clinical trials and they are associated with a delay in ampakines reaching plasma therapeutic levels (>1 hour). Thus, a drug therapy that rapidly reduces OIRD without interfering with the desired analgesia and induction of significant side-effects remains an unmet clinical need.

Opioids are not the only class of drugs that cause respiratory depression. Propofol, alcohol, barbiturates and benzodiazepines can all cause respiratory depression by suppressing respiratory rhythmogenesis and drive to cranial and spinal motoneurons (Ren and Greer, 2006; Ren et al., 2012, 2013). A significant part of the mechanism of action of these agents is via modulation of GABAA receptor conductances located on preBötC neurons and respiratory motoneurons. Besides opioid and non-opioid drugs, other mechanisms or causes of respiratory depression include obstructive sleep apnea, central sleep apnea, apnea of prematurity, Prader-Willi Syndrome, Rett Syndrome, Pompe Disease, Cheyne-Stokes breathing, neuronal degeneration, stroke, heart failure, brain trauma, Parkinson's Disease, and spinal cord injury.

Nicotinic receptors are made up of five subunits, arranged symmetrically around a central pore (Gotti et al., 2006). There have been reports of nAChRs in the brainstem where respiratory networks are located and on carotid chemoreceptors that modulate breathing. (Wada et al., 1989; Shao and Feldman, 2002; Chamberlin et al., 2002; Liu et al., 2005; Hatori et al., 2006). This includes α7, α4, α3, β4, and β2 subunits.

Accordingly, there is a need in the art to elucidate the relative efficacy of the above compounds for countering opioid-induced, non-opioid-induced, or other causes of respiratory depression.

### SUMMARY OF THE INVENTION

The present invention relates to methods of using compounds which target neuronal heteromeric nicotinic acetylcholine receptors for treating, preventing, or ameliorating respiratory depression and overdose induced by an opioid, or other causes of respiratory depression in a subject.

In one aspect, the invention comprises a method of treating, preventing, or ameliorating respiratory depression and overdose induced by an opioid, or other causes of respiratory depression in a subject, comprising administering to the subject an effective amount of a compound capable of activating a neuronal heteromeric nicotinic acetylcholine receptor or a composition comprising same.

In one embodiment, the other cause of respiratory depression is selected from a non-opioid drug, obstructive sleep apnea, central sleep apnea, apnea of prematurity, hypoxia, Prader-Willi Syndrome, Rett Syndrome, Pompe Disease, Cheyne-Stokes breathing, neuronal degeneration, stroke, heart failure, brain trauma, Parkinson's Disease, or spinal cord injury. In one embodiment, the non-opioid drug is selected from propofol, isoflurane, a barbiturate, a benzodiazepine, a volatile anesthetic, or alcohol.

In one embodiment, the respiratory depression and the overdose, or the other cause of respiratory depression is treated, prevented, or ameliorated with oral, nostril spray, intravenous or intramuscular administration of the compound.

In one embodiment, the compound is selected from a positive allosteric modulator or nicotinic acetylcholine agonist selected from a full agonist or a partial agonist that acts at α4β2 nAChRs. In one embodiment, the full agonist comprises 3-(2(s)-azetidinylmethoxy) pyridine (A85380). In one embodiment, the partial agonist comprises (E)-N-Methyl-4-(3-pyridinyl)-3-butene-1-amine (Rivanicline). In one embodiment, the positive allosteric modulator comprises 3-[3-(3-Pyridinyl)-1,2,4-oxadiazol-5yl]benzonitrile (NS9283).

In one embodiment, the neuronal heteromeric nicotinic acetylcholine receptor is a WIN and potentially other types of β2 containing nicotinic acetylcholine receptor. In one embodiment, the neuronal heteromeric nicotinic acetylcholine receptor is a β4 containing nicotinic acetylcholine receptor.

In another aspect, the invention comprises a method of activating a neuronal heteromeric nicotinic acetylcholine receptor in a cell or organism, comprising exposing the cell or the organism to the above compound. In one embodiment, the cell comprises a neuronal cell.

In yet another aspect, the invention comprises a method of inducing analgesia, anesthesia, or sedation in a subject, while simultaneously treating, preventing, or ameliorating respiratory depression and overdose induced by an opioid, or other causes of respiratory depression, comprising administering to the subject an effective amount of a compound capable of activating a neuronal heteromeric nicotinic acetylcholine receptor or a composition comprising same.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a schematic diagram of a prior art method (Ren et al., 2015) applied to evaluate effects of α4β2 nAChR compounds on fentanyl-induced respiratory depression, analgesia, and sedation in adult rats.

FIGS. 2A-E are traces of respiratory rate from whole-body plethysmograph recordings following administration of vehicle (saline) (FIG. 2A), A85380 (FIG. 2B), Rivanicline (FIG. 2C), NS9283 (FIG. 2D), and PNU282987 (FIG. 2E).

FIG. 2F is a graph of population data showing the time course of changes of respiratory frequency in response to fentanyl with the subsequent administration of vehicle (saline), A85380, and Rivanicline.

FIG. 2G is a graph of population data showing the time course of changes of respiratory frequency in response to fentanyl with the subsequent administration of vehicle (HPCD), NS9283, and PNU282987.

FIGS. 3A-D are recordings of rectified and integrated discharge from the fourth cervical nerve, showing the effects of A85380 (25 nM), Rivanicline (0.5 μM), NS9283 (15 μM), and PNU282987 (1-20 μM) on respiratory rhythm generated by brainstem-spinal cord in vitro preparations.

FIGS. 3E-F are graphs of population data.

FIGS. 3G-J are recordings of rectified and integrated discharge from the fourth cervical nerve, showing the effects of A85380, Rivanicline, NS9283, and PNU282987 following bath application of DAMGO.

FIGS. 3K-L are graphs of population data.

FIGS. 4A-B are recordings of rectified and integrated discharge from the hypoglossal nerve (XIIn) in medullary slice and preBötC, showing the effects of A85380 (25 nM) and reversal of such effects by DHβE (200 nM) (FIG. 4A); and reversal of the effects of DAMGO by A85380 (FIG. 4B).

FIGS. 4C-D are graphs of population data.

FIG. 5 shows recordings of rectified and integrated discharge from the fourth cervical nerve, showing the effects of A85380 (25 nM) in the presence of ethanol (50 mM) and pentobarbital (50 μM) on respiratory rhythm generated by brainstem-spinal cord in vitro preparations.

FIGS. 6A-B are traces of respiratory activity from whole-body plethysmograph recordings following co-administration of A85380 and propofol.

FIG. 7 shows recordings of rectified and integrated discharge from the hypoglossal nerve (XIIn), showing the effects of A85380 (25 nM) in the presence of lower than normal extracellular potassium levels (6 mM) on respiratory rhythm and motor output generated by medullary slice in vitro preparations.

FIG. 8 relates to weak respiratory drive to hypoglossal motoneurons that occurs in obstructive and central apnea and apnea of prematurity and shows recordings of rectified and integrated discharge from the hypoglossal nerve (XIIn) from brainstem-spinal cord cut at the level of C2: an initial strong respiratory activity within 40 min, followed by period of slow respiratory frequency and weak motor output (small amplitude and short bursting duration), and increased respiratory frequency, amplitude and bursting duration after bath application of A85380 (25 nM).

FIG. 9 is related to spinal cord injury and shows a diagram of a brainstem-spinal cord preparation with C2 hemisection (left), and rectified and integrated signals made from C4 recordings from a P0 rat brainstem-spinal cord preparation showing the respiratory activity both contralateral (A: top traces) and ipsilateral (B: bottom traces) to one side C2 hemisection.

FIGS. 10A-D are representative whole body plethysmographic recordings from four postnatal day 3 pups showing the effects of sazetidine-A (0.5 mg/kg) and VMY-2-95 (1 mg/kg) on fentanyl-induced respiratory depression in newborn rats.

FIGS. 10E-G are graphs of population data showing respiratory frequency (f_{R}), tidal volume (V_{T}), and minute ventilation (V_{E}) relative to control prior to fentanyl administration: dose-dependent alleviation of fentanylinduced respiratory depression by sazetidine, and VMY-2-95, with both effects blocked by α4β2, α6β2, α4β4 antagonist DEβE.

FIGS. 11A-C show that the administration of vehicle (HPCD, iv bolus, approximately 7 min after fentanyl) had no effect on fentanyl-induced respiratory depression (FIG. 11A), administration of sazetidine-A (1 mg/kg, iv bolus, approximately 7 min after fentanyl) had no effect on fentanyl-induced respiratory depression (FIG. 11B), and administration of VMY-2-95 (1 mg/kg, iv bolus) reversed the fentanyl-induced decrease of respiratory rate (FIG. 11C).

FIG. 11D is population data showing of sazetidine-A (0.5-2 mg/kg) had no effects on fentanyl-induced decrease in respiratory rate (relative to control prior to fentanyl infusion).

FIG. 11E is population data showing of VMY-2-95 (1 mg/kg)-induced alleviation of fentanyl-induced decrease in respiratory rate (relative to control prior to fentanyl infusion).

FIGS. 12A and 12B are representative whole body plethysmographic recordings from two pups. FIG. 12A shows that administration of fentanyl (35 μg/kg) co-administered with saline vehicle caused a marked depression of respiratory frequency and a mild depression of tidal volume within 7 min post fentanyl administration and subsequent administration of SIB 1553A (40 mg/kg) partially reversed fentanyl-induced decrease in respiratory rate (f_{R}), without marked effects on fentanyl-induced decrease in tidal volume (V_{T}), respectively. FIG. 12B shows that administration of fentanyl (35 μg/kg) co-administered with non-selective nicotinic receptor antagonist mecamylamine (Mec, 6 mg/kg) caused a marked depression of respiratory frequency and a mild depression of tidal volume within 7 min post fentanyl administration and subsequent administration of SIB 1553A (40 mg/kg) had no effects on fentanyl-induced respiratory depression.

FIG. 12C is population data showing f_{R} relative to control prior to fentanyl administration.

FIGS. 13A and 13B are representative whole body plethysmographic recordings from two pups. FIG. 13A shows that administration of fentanyl (35 μg/kg) co-administered with saline vehicle caused a marked depression of respiratory frequency and a mild depression of tidal volume within 7 min post fentanyl administration and that subsequent administration of lobeline (10 mg/kg) partially reversed fentanyl-induced decrease in respiratory rate (f_{R}), and completely reversed fentanyl-induced decrease in tidal volume (V_{T}). FIG. 13B shows that administration of fentanyl (35 μg/kg) co-administered with non-selective nAChR receptor antagonist mecamylamine (6 mg/kg) did not affect the fentanyl-induced respiratory depression and subsequent administration of lobeline (10 mg/kg, 7 min post fentanyl) had less effects on fentanyl-induced respiratory depression.

FIGS. 13C-E is population data showing f_{R}, V_{T}, and minute ventilation (V_{E}) relative to control prior to fentanyl administration.

FIGS. 14A and 14B show that administration of fentanyl (30 μg/kg over 10 min, iv infusion) caused a marked respiratory depression (f_{R}, V_{T}, and V_{E}) in whole body plethysmographic recordings from two rats. Subsequent administration of saline vehicle (iv) had no effect on fentanyl-induced respiratory depression (FIG. 14A) and subsequent administration of lobeline (3 mg/kg, iv bolus, approximately 7 min after fentanyl) completely reversed fentanyl-induced respiratory depression (FIG. 14B).

FIGS. 14C-E is population data showing respiratory parameters (f_{R}, V_{T}, V_{E}) relative to control prior to fentanyl infusion.

FIG. 14F is population data showing arterial oxygen saturation (Sao₂).

FIG. 15 shows the effects of lobeline on the heart rate in adult rats. Data point shown 2 min post saline (iv, n=5), 2 min post lobeline (3 mg/kg, iv, n=5), 7 min post fentanyl (30 μg/kg over 10 min iv infusion), and 2 min after subsequent administration of saline (n=8) or lobeline (n=8).

FIGS. 16A-C show that co-administration of fentanyl (12 μg/kg over 1 min, iv infusion) with saline vehicle (1 min iv infusion) caused a marked respiratory depression (f_{R}, V_{T}, and V_{E}) and apneas in two rats (FIGS. 16A-B) and that co-administration of fentanyl (12 μg/kg over 1 min, iv infusion) with lobeline (3 mg/kg over 1 min, iv infusion) markedly prevented fentanyl-induced respiratory depression (f_{R}, V_{T}, and V_{E}) and abolished apneas (FIG. 16C).

FIGS. 17A and 17B show the effects of lobeline on the time spent engaging in nociceptive behaviors (licking and lifting), where FIG. 17A is Phase I (0-5 minutes post formalin injection) and FIG. 17B is Phase II (20-40 minutes post formalin injection).

FIGS. 18A-C shows that bath application of nicotine (600 nM) or A85380 (25 nM) markedly increased baseline respiratory frequency (f_{R}) without effects on respiratory amplitude, duration, and area in neonatal brainstem-spinal cord preparations. PNU282987 (30 μM) slightly increased baseline f_{R}.

FIGS. 18D-F show DAMGO (200 nM) suppressed f_{R} and burst area, the effects were reversed by a subsequent application of nicotine (600 nM), A85380 (25 nM), but not by PNU282987 (30 μM).

FIG. 18G shows that co-administration of DAMGO and α4β2 antagonist DHβE (400 nM) suppressed f_{R} and burst area; the effects were no longer affected by a subsequent application of nicotine (600 nM).

FIGS. 18H-I is population data showing baseline f_{R} and DAMGO-induced respiratory depression (relative to control).

FIG. 19A shows that bath application of A85380 increased baseline respiratory frequency and decreased baseline respiratory burst area.

FIG. 19B shows that bath application of DAMGO (200 nM) suppressed respiratory frequency and burst area; the effects were reversed by a subsequent application of A85380.

FIG. 19C is population data.

FIGS. 20A-F are representative whole body plethysmographic recordings from six postnatal day 3 pups. Administration of fentanyl (35 μg/kg, FIGS. 20A-D) or co-administration with DHβE (6 mg/kg, FIGS. 20E-F) caused a marked depression of respiratory frequency and a mild depression of tidal volume within 7 min post fentanyl administration. Subsequent administration of saline had no effect on fentanyl-induced respiratory depression (FIG. 20A). Nicotine (0.6 mg/kg, FIG. 20B) and A85380 (0.06 mg/kg, FIG. 20C), but not PNU282987 (20 mg/kg, FIG. 20D) reversed fentanyl-induced respiratory depression. However, neither nicotine (0.6 mg/kg, FIG. 20E) nor A85380 (0.06 mg/kg, FIG. 20F) had any effect on respiratory depression induced by fentanyl co-administrated with DHβE (6 mg/kg).

FIGS. 20G-I is population data showing respiratory frequency (f_{R}), tidal volume (V_{T}), and minute ventilation (V_{E}) relative to control prior to fentanyl administration.

FIGS. 21A-D are representative whole body plethysmographic recordings from 4 adult rats. Administration of fentanyl (60 μg/kg over 20 min, iv infusion) caused a marked decrease of respiratory rate within 7 min of the infusion. The fentanyl-induced decrease of respiratory frequency was not affected by subsequent administration (iv) of saline (FIG. 21A) or PNU282987 (10 mg/kg, FIG. 21D), but was diminished by nicotine (0.3 mg/kg, FIG. 21B) and A85380 (0.03 mg/kg, FIG. 21C).

FIGS. 21E-G is population data showing the time course of changes of respiratory frequency relative to control prior to drug administration.

FIGS. 22A and 22B are representative whole body plethysmographic recordings from 2 adult rats where 2 min after saline (neck subcutaneously), administration of fentanyl (20 μg/kg over 400s, iv infusion) caused a marked depression of respiratory frequency and minute ventilation (FIG. 22A) and pre-administration of A85380 (0.06 mg/kg, subcutaneously) 2 min prior to fentanyl reduced the fentanyl-induced decrease of respiratory frequency (FIG. 22B).

FIG. 22C is population data showing respiratory frequency relative to control prior to drug administration.

FIGS. 23A and 23B are representative whole body plethysmographic recordings from 2 adult rats, where a bolus of remifentanil (5 μg/kg iv bolus over 20s, co-administrated with saline) caused marked apneas and decreased minute ventilation (V_{E}) in the first minute (FIG. 23A) and co-administration of A85380 (0.06 mg/kg, iv) with remifentanil markedly reduced the remifentanil-induced apneas and decrease in V_{E} (FIG. 23B).

FIGS. 23C-D is population data.

FIG. 24 provides a graphic outline of the experimental protocol. A85380 (0.06 mg/kg, neck subcutaneously, sc) or saline was administrated 2 min prior to fentanyl (20 μg/kg over 400 s, iv infusion). In particular, in one set of animals, the righting reflex testing started 10 min post-fentanyl, and then the animal was removed from the chamber for thermal nociception testing 40 min post-fentanyl (FIG. 24A). In another set of animals, formalin was administered 10 min post-fentanyl (FIG. 24B).

FIG. 25A shows the effects of A85380 on paw withdrawal latency in response to thermal stimuli, measured at 42 min after A85380 or saline W/O subsequent fentanyl infusion.

FIG. 25B shows the effects of A85380 on the time spent engaging in nociceptive behaviors (licking and lifting) 20-40 minutes post formalin, measured at 32-52 min after A85380 or saline W/O subsequent fentanyl infusion.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise.

The present invention relates to methods of using compounds which target neuronal heteromeric nicotinic acetylcholine receptors for treating, preventing, or ameliorating respiratory depression and overdose induced by an opioid, or other causes of respiratory depression in a subject.

As used herein, the abbreviation “neuronal” refers to neurons or nerve cells which are electrically excitable cells that receive, process, and transmit information through electrical and chemical signals. Neurons are the primary components of the central nervous system, which includes the brain and spinal cord, and of the peripheral nervous system, which comprises the autonomic nervous system and the somatic nervous system.

As used herein, the term “neuronal heteromeric nicotinic acetylcholine receptors” (abbreviated as “neuronal nAChR” or “neuronal nAChRs”) refers to pentamers of heteromeric combinations of a (2-10) and β (2-4) subunits found in neurons which have different pharmacological and biophysical properties and locations (Gotti et al., 2006). The β2 and the β4 nicotinic acetylcholine receptor (nAChR) subunits are expressed throughout the central nervous system and the peripheral nervous system. These two β subunits can form heteromultimeric channels with any of the α2, α3, α4, or α5 subunits. α4β2 are among the most abundant in the mammalian brain, whereas α3β4 primarily in peripheral ganglia. Neuronal heteromeric nicotinic acetylcholine receptors require different subunit assembly partners (at least one a plus at least one β subunits), including, but not limiting to α2β4, α3β4, α4β4, α2β2, α3β2, α4β2, α6β2, α7β2, α4α5β2, and α4α6β2 nicotinic acetylcholine receptors.

As used herein, the term “compound” refers to a substance which targets neuronal heteromeric nicotinic acetylcholine receptors. In one embodiment, the compound is selected from a nicotinic acetylcholine agonist or a positive allosteric modulator.

As used herein, the term “agonist” refers to a compound which binds to a receptor and activates the receptor to produce a biological response. As used herein, the term “nicotinic acetylcholine agonist” refers to a compound which mimics the action of acetylcholine at nicotinic acetylcholine receptors. In one embodiment, the nicotinic acetylcholine agonist comprises a full agonist. As used herein, a “full agonist” binds and activates a receptor with an efficacy equal to the endogenous agonist. In one embodiment, the full agonist comprises 3-(2(s)-azetidinylmethoxy) pyridine (abbreviated as “A85380” and developed by Abbott Laboratories). A85380 shows selectivity for the α4β2 or α6β2 nicotinic acetylcholine receptors. Further, A85380 is a broad spectrum analgesic at doses (0.02-0.06 mg/kg) which do not induce marked behavioral effects (Sullivan et al., 1996; Curzon et al., 1998; Rueter et al., 2000; Rueter et al., 2003; Rueter et al., 2006). Radiolabelled forms of A85380 are safe in humans (Rueter et al., 2006). In one embodiment, the full agonist is 2-((2R,6S)-6-((S)-2-Hydroxy-2-phenylethyl)-1-methylpiperidin-2-yl)-1-phenylethanone, which is commonly referred to as “lobeline”. Lobeline is a full agonist of α4β4 human nAChR, partial agonist of α4β2 human nAChR, and partial agonist of α3β4 rat nAChR (Wu et al., 2006; Kaniakova et al., 2014).

In one embodiment, the nicotinic acetylcholine agonist comprises a partial agonist. As used herein, a “partial agonist” binds and activates a receptor with less efficiency than the endogenous agonist. In one embodiment, the partial agonist comprises (E)-N-Methyl-4-(3-pyridinyl)-3-butene-1-amine or (E)-metanicotine, which is commonly referred to as “Rivanicline” (co-developed by Targacept and Falk Pharmaceuticals). Rivanicline shows selectivity for the α4β2, α6β2 nicotinic acetylcholine receptors. Further, Rivanicline is a broad spectrum analgesic at doses (3-10 mg/kg) which do not induce marked side effects in rodents (Lippiello et al., 1996; Damaj et al., 1999). In one embodiment, the partial agonist of α4β2, α6β2 nicotinic acetylcholine receptors comprises 6-[5-[(2S)-2-Azetidinylmethoxy]-3-pyridinyl]-5-hexyn-1-ol, which is commonly known as “sazetidine-A”, and its analog 3-[(2S)-2-Azetidinylmethoxy]-5-(2-phenylethynyl)-pyridine, which is commonly known as “VMY-2-95”. In one embodiment, the partial agonist is (±)-4-[2-((N-methyl)-2-pyrrolidinyl)ethyl]thiophenol, which is commonly known as “SIB-1533A” and which is a partial agonist for α4β2 nicotinic acetylcholine receptors but a full agonist for (34 containing selective subtype nicotinic acetylcholine receptors such as α2β4 nicotinic acetylcholine receptors.

As used herein, the term “positive allosteric modulator” refers to a compound which induces an amplification of the effect of a primary ligand that directly activates or deactivates the function of a target protein. Positive allosteric modulators of the present invention are allosteric modulators of neuronal heterogenic nicotinic acetylcholine receptors which indirectly increase the activity of the receptors. In one embodiment, the positive allosteric modulator (PAM) comprises 3-[3-(3-Pyridinyl)-1,2,4-oxadiazol-5yl]benzonitrile, which is commonly referred to as “NS9283” (developed by Neurosearch Inc.). NS9283 was developed as an analgesic (Lee et al., 2011; Pandya et al., 2011; Zhu et al., 2011; Rode et al., 2012; Timmermann et al., 2012; Grupe et al., 2013; Olsen et al., 2013). NS9283 does not have any intrinsic activity on the nicotinic acetylcholine receptors per se, but rather amplifies the effects of acetylcholine binding by slowing the rate of deactivation. NS9283 (2.5-30 mg/kg) does not induce side-effects in rodents (Lee et al., 2011; Zhu et al., 2011; Timmermann et al., 2012). NS9283 is a positive allosteric modulator of (α4)₃(β2)₂ nAChR.

Other nicotinic acetylcholine full agonists, partial agonists and PAMS which may be useful in the present invention include, but are not limiting to, 3-bromcytisine, 3-pyr-Cytisine, 5-Iodo-A-85380, 2-Methyl-3-{[(2S)-pyrrolidin-2-yl]methoxy}pyridine (ABT 089, pozanicline), 1-pyridin-3-ylpyrrolidin-3-amine (ABT202), ABT418 (ebanicline), ABT594 ([(R)-5-(2-azetidinylmethoxy)-2-chloropyridine], tebanicline), (1S,5S)-3-(5,6-Dichloro-3-pyridinyl)-3,6-diazabicyclo[3.2.0]heptane (ABT894, sofinicline), 2S)-3-ethynyl-5-(1-methylpyrrolidin-2-yl)pyridine (Altinicline, SIB-1508Y), AVE 3183, AZD1446 (TC6683), (2S,4E)-5-(5-isopropoxypyridin-3-yl)-N-methylpent-4-en-2-amine (AZD-3480, Ispronicline, TC-1734), cannabidiol, CC4, CP-601927, cytisine, (5aS,8S,10aR)-5a,6,9,10-Tetrahydro,7H,11H-8,10a-methanopyrido[2′,3′:5,6]pyrano[2,3-d]azepine (Dianicline, SSR-591,813), Desformylflustrabromine (2-[6-bromo-2-(2-methylbut-3-en-2-yl)-1H-indol-3-yl]-N-methylethanamine), Dimethylphenylpiperazinium (DMPP), nicotine, RJR 2429, TC 1827, TC 2216, TC 2559, TC 2696, TC 6499, TC 8831, TI-10165, UB-165, and 7,8,9,10-Tetrahydro-6,10-methano-6H-pyrazino[2,3-h] [3]benzazepine (Varenicline, Chantix, Champix).

Certain embodiments of the invention relate to methods and uses of the above compounds which target neuronal heteromeric nicotinic acetylcholine receptors for treating, preventing, or ameliorating respiratory depression and overdose induced by an opioid, or other causes of respiratory depression in a subject. In one embodiment, the invention comprises a method of treating, preventing, or ameliorating respiratory depression and overdose induced by an opioid, or other causes of respiratory depression in a subject, comprising administering to the subject an effective amount of one the above compounds to the subject. In one embodiment, the invention comprises use of one of the above compounds to treat, prevent, or ameliorate respiratory depression and overdose induced by an opioid, or other causes of respiratory depression in a subject.

In one embodiment, the invention comprises a method of activating a neuronal heteromeric nicotinic acetylcholine receptor in a cell or organism, comprising exposing the cell or the organism to the above compound. In one embodiment, the cell comprises a neuronal cell.

In one embodiment, the invention comprises a method of inducing analgesia, anesthesia, or sedation in a subject, while simultaneously treating, preventing, or ameliorating respiratory depression and overdose induced by an opioid, or other causes of respiratory depression, comprising administering to the subject an effective amount of a compound capable of activating a neuronal heteromeric nicotinic acetylcholine receptor or a composition comprising same.

As used herein, the term “respiratory depression” refers to a variety of conditions characterized by reduced respiratory frequency and inspiratory drive to cranial and spinal motor neurons. Specifically, respiratory depression refers to conditions where the medullary neural network associated with respiratory rhythm generating activity does not respond to accumulating levels of PCO₂ (or decreasing levels of PO₂) in the blood and subsequently under-stimulates motoneurons controlling lung musculature. The term is meant to include other causes of respiratory depression associated with anesthetics (for example, propofol, isoflurane), barbiturates, benzodiazepines, alcohol, apnea of prematurity, genetic disorders (for example, Rett Syndrome, Pompe Disease), Cheyne-Stokes breathing and neurological disorders (for example, stroke, trauma, degenerative diseases that affect the brainstem).

As used herein, the term “opioid” is meant to include a drug, hormone, or other chemical or biological substance, natural or synthetic, having a sedative, narcotic, or otherwise similar effect(s) to those containing opium or its natural or synthetic derivatives. A non-exclusive list of opioids includes alfentanil, allylprodine, alphaprodine, anileridine, benzylmorphine, bezitramide, buprenorphine, butorphanol, carfentanil, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine. The definition includes all opioids, from any source, including naturally-derived compounds, synthetic compounds, and semi-synthetic compounds. The definition also includes all isomers, stereoisomers, esters, ethers, salts, and salts of such isomers, steroeisomers, esters, and ethers, whenever the existence of such isomers, stereoisomers, esters, and ethers is possible within the specific chemical designation. In one embodiment, the opioid is fentanyl.

As used herein, the term “subject” refers to any member of the animal kingdom. In one embodiment, a subject is a human patient. In one embodiment, a subject is an adult patient. In one embodiment, a pediatric patient is a patient under 18 years of age, while an adult patient is a patient 18 years of age or older.

Compounds of the present invention may be formulated for therapeutic use. A composition or a pharmaceutical composition may comprise a compound of the present invention in combination with one or more pharmaceutically acceptable carriers. As used herein, the term “carrier” means a suitable vehicle which is biocompatible and pharmaceutically acceptable, including for instance, liquid diluents which are suitable for administration. As used herein, the term “biocompatible” means generating no significant undesirable host response for the intended utility. Most preferably, biocompatible materials are non-toxic for the intended utility. Thus, for human utility, biocompatible is most preferably non-toxic and otherwise non-damaging to humans or human tissues. Those skilled in the art are familiar with any pharmaceutically acceptable carrier that would be useful in this regard, and therefore the procedure for making pharmaceutical compositions in accordance with the invention will not be discussed in detail. As used herein, the term “pharmaceutically acceptable” means a substance which does not significantly interfere with the effectiveness of the compound, and which has an acceptable toxic profile for the host to which it is administered.

Suitably, pharmaceutical compositions comprising a compound of the present invention may in various embodiments be formulated for administration parenterally in dosage unit formulations containing conventional non-toxic pharmaceutically acceptable carriers, adjuvants, and vehicles. The term “parenteral” as used herein includes subcutaneous injections, nostril spray, intradermal, intravenous, intramuscular, intravascular, intrasternal, intrathecal injection or infusion techniques. In one embodiment, a compound of the present invention is administered intramuscularly.

The pharmaceutical compositions may be in the form of a sterile injectable aqueous suspension. This suspension may be formulated according to known art using those suitable dispersing or wetting agents and suspending agents. The sterile injectable preparation may also be a sterile injectable solution or a suspension in a non-toxic parentally acceptable diluent or solvent. Among the acceptable vehicles and solvents that may be employed are water, Ringer's solution, and isotonic sodium chloride solution. In addition, sterile, fixed oils may be employed as a solvent or suspending medium. Adjuvants such as local anesthetics, preservatives, and buffering agents may optionally also be included in the injectable solution or suspension.

Useful dosages of a compound of the present invention depend upon many factors that are well known to those skilled in the art, for example, the type and pharmacodynamic characteristics of the compound; age, weight and general health condition of the subject; nature and extent of symptoms; any concurrent therapeutic treatments; frequency of treatment and the effect desired.

As used herein, the term “effective amount” means any amount of a formulation of a compound of the present invention useful for treating, preventing, or ameliorating respiratory depression and overdose induced by an opioid, or other causes of respiratory depression in a subject upon administration. An effective amount of the composition provides either subjective relief of symptoms or an objectively identifiable improvement as noted by the clinician or other qualified observer. As used herein, the terms “treating,” “preventing” and “ameliorating” refer to interventions performed with the intention of alleviating the symptoms associated with, preventing the development of, or altering the pathology of a disease, disorder or condition. Thus, in various embodiments, the terms may include the prevention (prophylaxis), moderation, reduction, or curing of a disease, disorder or condition at various stages. In various embodiments, therefore, those in need of therapy/treatment may include those already having the disease, disorder or condition and/or those prone to, or at risk of developing, the disease, disorder or condition and/or those in whom the disease, disorder or condition is to be prevented.

In the development of the invention, it was discovered that in one embodiment using compounds which specifically target α4β2 subtype of nicotinic acetylcholine receptors is a potent, effective method of overcoming opioid-induced respiratory depression. While many of these compounds have been examined in pre-clinical and clinical trials of non-respiratory related functions involving α4β2 nAChR (e.g. analgesia, cognition, smoking cessation), the relative efficacy of these compounds for countering opioid-induced respiratory depression had not yet been discovered. The compounds readily cross the blood-brain barrier and act specifically on α4β2 nicotinic acetylcholine receptors. Further, the half-life of the compounds in plasma/brain in the rat is approximately 1-2 hours (Rueter et al., 2006; Bencherif et al., 1997; Timmermann et al., 2012).

The data presented in the Examples below show a clear and robust reversal of opioid-induced respiratory depression by compounds modulating activity of neuronal heteromeric nicotinic acetylcholine receptors, such as α4β2 or β4 containing nicotinic acetylcholine receptors, whereas compounds targeting α7 nicotinic acetylcholine receptors expressed by respiratory neurons fail to do so. The compounds can be used clinically without marked side-effects. In contrast, compounds which target α3β4 nicotinic acetylcholine receptors have gastrointestinal side effects and those targeting α7 nicotinic acetylcholine receptors may cause nicotine-induced seizures and cell proliferation and angiogenesis in some cancers (Jain, 2004; Damaj et al., 1999; Iha et al., 2017; Egleton et al., 2008; Paleari et al., 2009; Tournier et al., 2011).

Without being bound by any theory, it will be appreciated by those skilled in the art that the findings could extend to other causes of respiratory depression associated with anesthetics (for example, propofol, isoflurane), barbiturates, benzodiazepines, alcohol, apnea of prematurity, genetic disorders (for example, Rett Syndrome, Pompe Disease), Cheyne-Stokes breathing and neurological disorders (for example, stroke, trauma, degenerative diseases that affect the brainstem).

Activation of neuronal heteromeric nicotinic acetylcholine receptors by compounds that result in the positive activation of the receptor (directly or indirectly) may overcome opioid-induced respiratory depression, with the full agonist A85380 being the most potent. In one embodiment, the opioid comprises fentanyl. Fentanyl is a powerful analgesic used commonly for acute and chronic pain, and is prevalent in cases of accidental overdose. Without being bound by any theory, it will be appreciated by those skilled in the art that the findings with fentanyl could extend to other μ-opiate receptor agents including, but not limited to, morphine and oxycodone.

The clinical implications include benefiting a cohort of patients who cannot receive adequate analgesia due to their sensitivity to opioid-induced respiratory depression. Further, additional antidotes to combat lethal overdose caused by opioids (for example, fentanyl) may arise. The compounds have the additional beneficial property of being analgesics (Marubio et al., 1999; Bitner et al., 2000; Decker et al., 2004; Damaj et al., 2007). Thus, it may be possible that lower doses of fentanyl will be needed for achieving desired analgesia when co-administered with the compounds, resulting in fewer side-effects including respiratory depression and sedation.

Embodiments of the present invention are described in the following Examples, which are set forth to aid in the understanding of the invention and should not be construed to limit in any way the scope of the invention as defined in the claims which follow thereafter.

### Example 1—Effects of Co-Administration of Fentanyl and α4β2 nAChR Targeting Drugs In Vivo Using Adult Rats

Plethysmographic recordings from adult rats were used to examine co-administration of drugs targeting α4β2 nAChRs (A85380, Rivanicline, NS9283) with fentanyl to determine whether there may be a significant reduction in the degree of OIRD and lethal overdose. The level of analgesia was monitored simultaneously to assess if there may be additive effects of the nAChR targeting drugs and fentanyl.

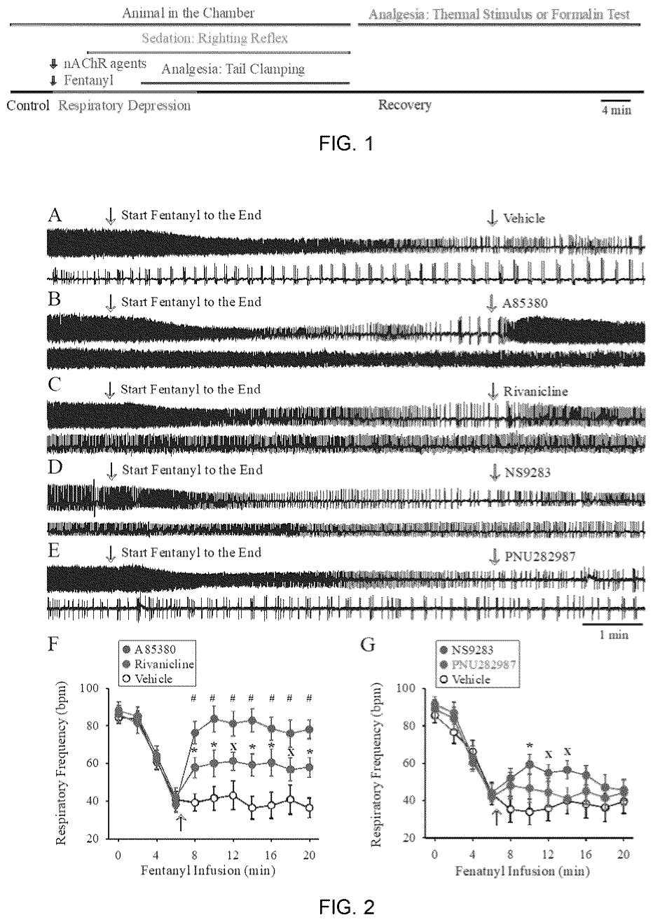

FIG. 1 is a schematic diagram of the experimental protocol (Ren et al., 2015). Briefly, three methods of nociceptive testing and a measure of sedation were performed in addition to measures of respiratory parameters. Righting reflex testing started 6-10 min after opioid administration and continued until the animal awoke from fentanyl-induced sedation. Tail clamping started 12 min after opioid infusion and was repeated every 4 mins. When the animal regained a righting reflex, it was taken out for either thermal or formalin testing. Thermal testing was repeated every 4 min until the paw withdrawal latency returned to control level. The formalin test was performed 0-40 min post formalin injection.

In greater detail, male Sprague-Dawley rats (300-500 g) were temporarily anesthetized with 3% isoflurane in an induction chamber during tail vein cannulation. The rat was placed within a plethysmograph modified to allow exteriorization of the tail for drug infusion. Pressure changes were recorded with a pressure transducer, signal conditioner, and an analogue-digital board (Axon). Measurements included frequency, number of apneas and relative changes in tidal volume and minute ventilation pre- and post-drug delivery (Ren et al., 2015). A pulse oximeter was placed on the tail to monitor arterial oxygen saturation levels and heart rate. Body temperature was measured using a rectal probe.

Fentanyl was infused into one tail vein (iv) and a bolus of α4β2 nAChR targeting drug was injected into the other tail vein. Fentanyl infusion commenced approximately 5 min after placement in the plethysmograph chamber. Suitable methodology for inducing various levels of fentanyl-induced RD and lethality are described in Ren et al. (2006) and Ren et al. (2009). Specifically, administration of 20 μg/kg delivered over 20 min suppresses respiratory frequency by <50% of control. Administration of 60 μg/kg fentanyl for the same duration suppresses respiratory frequency by >50% of control. A rapid infusion of 80 μg/kg fentanyl (delivered in 1 min) typically induces lethal apneas.

Doses of α4β2 nAChR targeting agents that lead to robust receptor activation were co-administered with various doses of fentanyl: A85380 (bolus 0.01, 0.03 mg/kg) (Sullivan et al., 1996; Curzon et al., 1998; Rueter et al., 2000; Rueter et al., 2003; Rueter et al., 2006); Rivanicline (bolus 3, 10 mg/kg) (Lippiello et al., 1996; Damaj et al., 1999); and NS9283 (bolus 10, 30 mg/kg) (Marubio et al., 1999; Timmermann et al., 2012; Maurer et al., 2017). Experiments using the α7 AChR agonist PNU282987 showed that activation of the α7 subtype does not markedly alleviate fentanyl-induced RD; thus, this receptor was not further investigated. The receptor antagonist dihydro-beta-erythroidine (DHβE; dissolved in saline; bolus 6 mg/kg) (Damaj et al., 1995) was administered to test the effects of blockade of endogenous activation of α4β2 nAChR. All nAChR targeting drugs are purchased from Tocris. Animals were tested as follows: 3 (fentanyl doses)×9 (co-administrations)×10-20 each group.

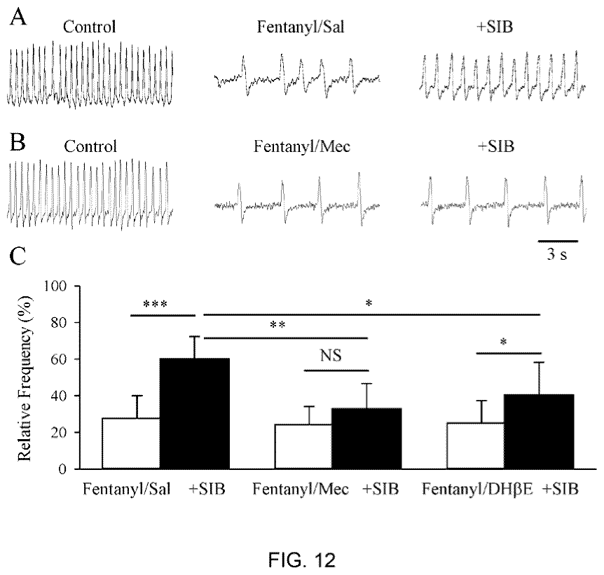

Data were expressed as mean±SEM for FIGS. 2-11; and mean±SD for FIGS. 12-25 (Sigmaplot). Randomization methods were used to assign units to experimental condition. Blind testing was used where one person administers the drug and a second person analyzes the data without knowledge of drug administration. For data passing the normality test (Shapiro-Wilk) and equal variance test (Brown-Forsythe), parametric statistics were used with t test for two groups or ANOVA for multiple groups followed by multiple comparisons. For data failing the normality test or equal variance test, non-parametric statistics were applied. p<0.05 was taken as significant difference.

In addition to respiratory parameters, the level of analgesia under the various conditions was examined by applying multiple methods of pain assessment, namely nociceptive testing (thermal, mechanical, and chemical) after co-administration of fentanyl and the α4β2 nAChR targeted agents to determine whether such agents (A85380, Rivanicline, NS9283) reduce nociceptive sensitivity and enhance opioid-induced analgesia.

The hot plate test measured thermal nociception by use of a plantar test apparatus consisting of an infrared heat source positioned directly beneath the hind paw, 20 mm below the chamber floor (Ren et al., 2006; 2009; 2015). When the rat perceived pain and withdrew its paw, the instrument automatically detected the withdrawal latency to the nearest 0.1s.

The mechanical test involved tail clamping with forceps (with consistent pressure). A positive response to tail clamping was indicated by obvious alterations in at least two of the following parameters: breathing variability (via plethysmography), heart rate (via pulse oximetry), oxygen saturation (via pulse oximetry), and body movement (visual observation) (Ren et al., 2015).

The formalin test examined nociceptive response via the injection of a dilute solution of formalin (50 μl, 1.5% formalin diluted in saline) in the intraplantar region of the right hind paw, followed by the scoring of time spent engaging in nociceptive behaviors (licking/lifting/flinching of the injected paw) in 5-minute interval blocks for the first phase (0-5 minutes; reflecting direct activation of nociceptors) and second phase (20-40 minutes; reflecting inflammation) of the assay (Dubuisson et al., 1977).

Previous studies have demonstrated that the analgesia induced after administration of 20 μg/kg and 60 μg/kg of fentanyl delivered over 20 min was lost at approximately 30 min and 100 min after the end of fentanyl infusion, respectively. Thus, the duration of analgesia with and without the addition of the various concentrations of α4β2 nAChR targeted agents was compared to assess whether analgesia might be enhanced with the combination of agents.

Changes in sedation and evidence for abnormal behavior were also monitored. In addition to respiratory depression, unintended sedation is another serious opioid-induced adverse event which contributes to patient morbidity and increased length of hospitalization (Jarzyna et al., 2011; Jungquist et al., 2013; Kessler et al., 2013). Although the underlying mechanisms are unclear, opioid-induced sedation is thought to involve the anticholinergic activity of opioids (Benyamin et al., 2008). Thus, it was determined whether activation of α4β2 might alleviate the analgesic opioid-induced sedation, providing an additional advantage for α4β2R targeting agents to counter opioid-induced respiratory depression. Sedation (loss of righting reflex) is defined as the rat's inability to right itself into the prone position after the animal has been placed supine.

Previous studies reporting minimal side-effects of α4β2 nAChR targeted agents were not performed with the co-administration of opiates. Ren et al. (2015) describes how to determine if serotonergic receptor agonists administered to reduce respiratory depression also generate marked unwanted behavioral changes (e.g., hyperalgesia, hyperventilation, abnormal body movements). Similar phenomena were not observed in the preliminary studies described herein with respect to α4β2 nAChR targeted agents (A85380, Rivanicline, NS9283).

To check whether α4β2 compounds alone affect the baseline nociceptive sensitivity and respiratory parameters, paw thermal withdrawal latencies were measured, followed by plethysmography for 30 min in control, and repeated 10 min post administration of α4β2 compounds (A85380 0.01, 0.03 mg/kg; Rivanicline 3, 10 mg/kg; NS9283 10, 30 mg/kg; DHβE 1 mg/kg; or vehicle; intraperitoneal ip injection). Nociceptive responses to formalin injection were also monitored. α4β2 compounds (ip) were administrated 20 min prior to formalin injection. Animals were tested as follows: 9 (treatments)×10 each group×2 nociception tests.

### Example 2—Efficacy of α4β2 nAChR Targeting Drugs (A85380, Rivanicline, or NS9283) to Rescue Respiratory Depression Induced by Fentanyl

In vivo experiments were conducted to determine if α4β2 nAChR targeting drugs may be used as rescue intervention against severe fentanyl-induced respiratory depression and lethality. The data show that all three drugs had some positive effect, with the full agonist A85380 exhibiting the most robust effect.

The advantage over naloxone is that analgesia was maintained. The protocol achieved a pronounced respiratory depression after 6 min of continuous fentanyl infusion (60 μg/kg for 20 min), or lethal apnea after 1 min of quick infusion (80 μg/kg for 1 min) into one tail vein. This was followed by administration of a bolus of α4β2 targeting drugs or vehicle injected into the other tail vein to assess the efficacy to rescue from respiratory depression.

It was determined whether intramuscular administration via thigh muscles might produce a positive alleviation of respiratory depression and relative timing compared to intravenous administration. In an emergency, it can be difficult to place an intravenous line; thus, a pre-packaged intramuscular injector similar to an Epi-Pen™ might offer distinct advantages. A85380 (0.01, 0.03 mg/kg), Rivanicline (3, 10 mg/kg), NS9283 (10, 30 mg/kg), or vehicle (saline, HPCD) were delivered (bolus via intravenous and intramuscular routes) subsequent to administration of fentanyl. The efficacy of rescue was compared with that achieved with naloxone (1 mg/kg). Animals were tested as follows: 2 (fentanyl doses)×9 (treatments)×2 (routes of administration)×10 each group.

Data from preliminary experiments on a small group of adult rats indicated that the agonists or positive allosteric modulator of α4β2 nAChRs alleviate fentanyl-induced respiratory depression. Representative traces are shown in FIGS. 2A-D. Marked reversal by activation of α7 nAChRs with the agonist PNU282987 (15 mg/kg) was not observed (FIG. 2E). Population data are shown in FIGS. 2F-G. Administration of the α4β2 nAChR full agonist A85380 (0.03 mg/kg, iv bolus) quickly and completely reversed the fentanyl-induced decrease of respiratory frequency over the whole period of fentanyl infusion. α4β2 nAChR partial agonist Rivanicline (3 mg/kg, iv bolus) quickly and partially reversed the fentanyl-induced decrease of respiratory frequency over the whole period of fentanyl infusion. α4β2 nAChR positive allosteric modulator NS9283 (10 mg/kg, iv bolus) partially and transiently reversed the fentanyl-induced decrease of respiratory frequency. No abnormal behavior was observed with any of these agents.

The data show that fentanyl caused a marked reduction in respiratory frequency that is reversed by α4β2 nAChR agents. Frequency is the primary respiratory parameter affected in humans exposed to opioids. The data also show a decrease in tidal volume after fentanyl infusion. Ren et al. (2006) reported that the reduced tidal volume is in part due to decreased drive to respiratory motoneurons and a larger component results from fentanyl-induced muscle rigidity and ribcage stiffness. The rigidity that occurs in rats is a well-documented phenomenon whose mechanism of action is poorly understood. Fentanyl-induced rigidity also occurs in some patients exposed to fentanyl but it is generally not nearly as pronounced as in rats. The α4β2 nAChR targeting drugs do not reduce the muscle rigidity and thus there is not a complete reversal of tidal volume. The in vivo model is thus not well-suited to assess whether or not the drugs are reversing any opioid-induced suppression of motoneuron activity. To address this aspect, in vitro preparations were used that allow for direct recordings from motoneurons (XII) and motor nerves (phrenic and XII) pre- and post-administration of opioids and α4β2 nAChR targeting drugs (Example 3).

FIGS. 2A-E show that α4β2 nAChR full agonist A85380, partial agonist Rivanicline, and positive allosteric modulator NS9283 alleviated fentanyl-induced decrease of respiratory rate in adult rats, but the α7 nAChR full agonist PNU282987 did not. Administration of fentanyl (60 μg/kg over 20 min, intravenous infusion) caused a marked decrease of respiratory rate. Traces are displayed continuously with whole-body plethysmograph recordings. Vehicle (saline) had no effect on fentanyl-induced decrease of respiratory rate (FIG. 2A). A85380 (0.03 mg/kg, iv bolus) very quickly reversed the fentanyl-induced decrease of respiratory rate (FIG. 2B). This is much faster than previous observations with ampakines. Rivanicline (3 mg/kg, iv bolus) quickly and partially reversed the fentanyl-induced decrease of respiratory rate (FIG. 2C). NS9283 (10 mg/kg, iv bolus) partially and transiently reversed the fentanyl-induced decrease of respiratory rate (FIG. 2D). PNU282987 (15 mg/kg, iv bolus) had no effect on the fentanyl-induced decrease of respiratory rate (FIG. 2E). Population data show the time course of changes of respiratory frequency in response to fentanyl with the subsequent administration of saline (n=7), A85380 (0.03 mg/kg, n=7), and Rivanicline (3 mg/kg, n=7) (FIG. 2F), and HPCD vehicle (n=8), NS9283 (10 mg/kg, n=6) or PNU282987 (1, 2, 3, 10, 15, 15 mg/kg, n=6 total) (FIG. 2G). (′p<0.05, *p<0.01, ^{#}p<0.001 relative to vehicle control). HPCD (2-hydroxypropyl-β-cyclodextrin) is a commonly used excipient to stabilize and solubilize pharmaceuticals that are insoluble in water. Two-way RM ANOVA followed by Holm-Sidak methods were used. The arrow demarcates the bolus of injection of nAChR agonists or vehicles. Neither A85380 (0.03 mg/kg, 97.8±3.1% of control, n=5), Rivanicline (3 mg/kg, 98.4±3.6% of control, n=5), NS9283 (10 mg/kg, 103.3±4.2% of control, n=4), nor PNU282987 (1, 3, 5, 10, 15 mg/kg, 102.9±4.1% of control, n=5 total) had marked effects on baseline minute ventilation, consistent with a previous finding (Lippiello et al., 1996).

### Example 3—Mechanistic Studies of the Interaction of μ-Opiate and α4β2 nACh Receptor Activation of preBötC Inspiratory Neurons and XII Motoneurons

Recordings of nerve activity and whole-cell patch recordings from in vitro preparations were used to examine the mechanisms of action of opiates and α4β2 nAChR targeting drugs on preBötC neurons and XII motoneurons. Medullary slice preparations that generate robust inspiratory rhythmic activity within preBötC and XII motoneuron populations were used (Funk, 2013). The medullary slices are cut at a specific rostrocaudal level to expose both the preBötC and XII motoneuron populations (Ruangkittisakul et al., 2006). The use of infrared differential-interference-contrast microscopy provides clear visualization of the neurons for targeting of the whole-cell patch electrodes.

Brainstem-spinal cord and medullary slice preparations were obtained from neonatal Sprague-Dawley rats. The rats were anesthetized with metofane, decerebrated, and the brainstem-spinal cord (BSSC) and medullary slice dissected (Smith et al., 1990; 1991). BSSC was continuously perfused with modified Krebs solution. A single rhythmic medullary slice was cut (500-650 μm thick) and continuously perfused with bathing solution. Inspiratory activity was recorded from the fourth ventral cervical nerve roots of BSSC or XII nerve rootlets of the medullary slice using an extracellular glass electrode.

Whole cell recordings targeted neurons located within about 70 mm of the slice surface. Intracellular pipettes were filled with an internal solution (in mM) containing 140 K-gluconate, 4 NaCl, 1 CaCl₂, 10 BAPTA, 10 HEPES, 5 MgATP, and 0.3 Na₃GTP (Ren et al., 2006). Input resistance was measured as Vm changes in response to hyperpolarizing current pulses. The frequency and amplitude of miniature EPSCs (mEPSCs) is measured and the effect of each drug was determined by comparing cumulative frequency histograms for changes in the average mEPSC frequency and amplitude values between periods of control and drug application (Loner et al., 2010).

The recordings will focus on rhythmic inspiratory neurons within the preBötC and motoneurons receiving inspiratory drive potentials in the XII nuclei to determine whether α4β2 nAChR targeting drugs (A85380, Rivanicline, NS9283) might have the following effects: 1) inducing a membrane depolarization in neurons that respond with membrane hyperpolarization and increases in input resistance to DAMGO in the presence and absence of tetrodotoxin (TTX 1 04); 2) modulating inspiratory drive potentials in preBötC inspiratory neurons in the absence and presence of DAMGO; 3) alleviating opioid-induced suppression of spontaneous EPSCs; and 4) acting via presynaptic mechanisms to alleviate opioid-induced suppression of miniature EPSCs (mEPSCs) in the presence of TTX (1 μM), strychnine (10 μM) and bicuculline (10 μM).

Data from previous medullary slice studies have determined that μ-opiate receptor activation suppresses transmitter release via pre-synaptic mechanisms within the preBötC and XII motoneuron nuclei (Lorier et al., 2010; Haji et al., 2003; Lalley et al., 2006; Ballanyi et al., 2010). There is also a selective postsynaptic action of μ-opiate agonists on preBötC neurons thought to be primarily involved in inspiratory rhythmogenesis (Gray et al., 2001; Montandon et al., 2011; Takeda et al., 2001; Loner et al., 2008). Activation of preBötC inspiratory neurons by nicotine and Rivanicline induces a tonic inward current associated with an increase in membrane noise, increase in the frequency and amplitude of spontaneous excitatory postsynaptic currents (EPSCs), and decreased amplitude of inspiratory drive current (Shao et al., 2002). Whether those pre/postsynaptic modulatory actions counter μ-opiate receptor mediated suppression of neuron excitability is unknown. Further, the effects of α4β2 nAChR targeting agents on inspiratory XII motoneurons have not yet been studied. An electrophysiological approach based on studies related to preBötC neurons (Montandon et al., 2011) and XII motoneurons (Lorier et al., 2010) was applied herein to determine whether α4β2 receptor targeting drugs might reverse μ-opiate receptor mediated suppression of neuronal excitability via presynaptic and/or postsynaptic mechanisms.

In vitro data indicate that the respiratory depression induced by the μ-opiate receptor agonist [D-Ala2, NMe-Phe4, Gly-ol5]-enkephalin (DAMGO) was alleviated by α4β2 nAChR targeting agents (A85380, Rivanicline, NS9283), similarly to what was observed with fentanyl in vivo with adult rats. DAMGO is typically used for in vitro experiments in the field rather than fentanyl which is used in vivo. They bind to the same μ-opiate receptor. The in vitro analyses were initiated by examining the effects of drugs added to the media bathing the brainstem-spinal cord that demonstrates inspiratory rhythmic discharge along the neuraxis (medulla and spinal cord).

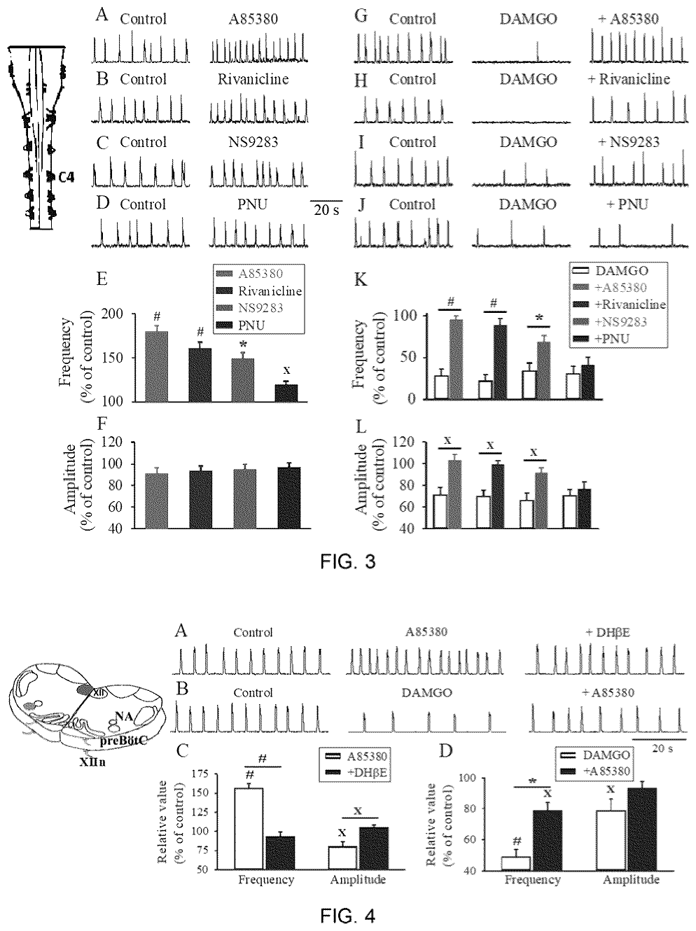

Recordings were made from rectified and integrated discharge from the fourth cervical nerve. As shown in FIGS. 3A-C (representative traces) and FIGS. 3E-F (population data; ^{x}p<0.05, *p<0.01, ^{#}p<0.001 relative to control with paired-t-test, or Wilcoxon signed rank test; n=6-8), α4β2 nAChR full agonist A85380, partial agonist Rivanicline, and positive allosteric modulator NS9283, at the concentrations applied, all increased baseline respiratory frequency without marked effects on the amplitude. Activation of the α7 AChR with the agonist PNU282987 (up to 20 μM, which has been shown to fully activate receptors in vitro) led to a very modest increase in respiratory frequency. FIGS. 3G-J (representative traces) and FIGS. 3K-L (population data; ^{x}p<0.05, *p<0.01, ^{#}p<0.001 between two compared groups, with one-way RM ANOVA followed by Holm-Sidak methods; each data point from n=6-8) showed a clear reversal of respiratory depression (decrease in respiratory frequency and amplitude) induced by DAMGO by subsequent bath application of A85380 (25 nM), Rivanicline (0.5 μM), and NS9283 (15 μM), but not by PNU282987 (1, 3, 10, 15, 15, 20 μM, n=6 total).

Recordings of rectified and integrated discharge were made from the hypoglossal nerve (XIIn) in the medullary slice (labeled in red) and the preBötC (labeled in blue). The medullary slice preparations containing the preBötC and XII motoneurons that discharge during the inspiratory phase of the respiratory cycle were used for a more direct assessment of drug action at the level of the preBötC. FIGS. 4A and C show that bath application of A85380 (25 nM) increased respiratory frequency and decreased the respiratory amplitude in the medullary slice. The effects are reversed by a subsequent bath application of α4β2 nAChR antagonist DHβE (200 nM). FIG. 4B shows that DAMGO (300 nM) induced suppression of respiratory frequency and amplitude that was alleviated by A85380 (25 nM). FIGS. 4C-D show population data (^{x}p<0.05, *p<0.01, ^{#}p<0.001, significant difference before and after drug was analyzed with one-way RM ANOVA followed by Holm-Sidak methods; n=6 for each data point).

### Example 4—Efficacy of α4β2 nAChR Targeting Drug (A85380) to Alleviate Respiratory Depression Induced by GABA_{A} Receptor Mediated Mechanisms

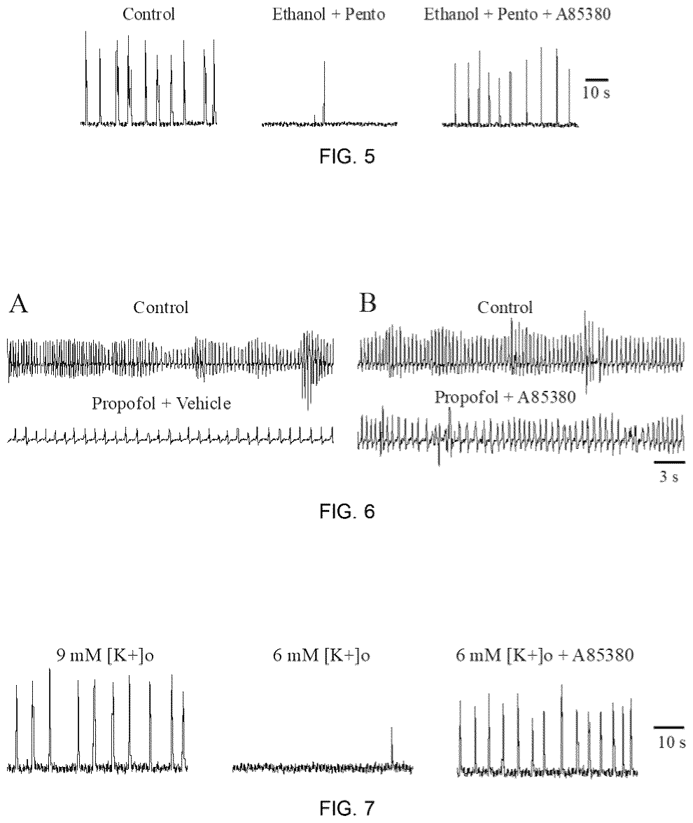

As shown in FIG. 5 of rectified and integrated C4 recordings, the addition of ethanol (50 mM) and pentobarbital (50 μM) to the medium bathing the newborn rat brainstem-spinal cord preparation caused a suppression of respiratory rhythm. The subsequent addition of A85380 (25 nM) alleviated the ethanol and pentobarbital-induced respiratory depression in the presence of ethanol and pentobarbital. Without being bound by any theory, targeting of α4β2 nAChRs may be a potent means of alleviating the effects of drugs causing respiratory depression via GABA_{A} receptor mechanisms in vitro.

In a further experiment, A85380 (0.03 mg/kg, ip) and propofol (30 mg/kg, ip) were co-administered in newborn rats in vivo (P3). In the control group, vehicle (saline) was administered with 30 mg/kg propofol (FIG. 6A). This caused a marked depression of respiratory frequency and amplitude within 5 minutes after propofol injection and lasted over 30 minutes. Co-administration of A85380 (0.03 mg/kg) with propofol (30 mg/kg) dramatically prevented propofol-induced respiratory depression (FIG. 6B). Without being bound by any theory, targeting of α4β2 nAChRs may be a potent means of alleviating the effects of drugs (e.g. propofol) causing respiratory depression via GABA_{A} receptor mechanisms in vivo.

### Example 5—Efficacy of α4β2 nAChR Targeting Drug (A85380) to Alleviate Respiratory Depression Caused by Weak Endogenous Excitatory Drive

Example 4 with opioids and GABAA receptor targeting drugs demonstrated respiratory depression induced by the effects of inhibitory actions on neurons. However, there is another general cause of respiratory depression which is caused by loss of normal excitatory input to the preBötC and respiratory motoneurons. This loss is thought to be responsible for obstructive and central apnea in some patients. The loss of excitatory drive is one mechanism by which XII motoneuron and preBötC neuron is suppressed in genetic disorders such as Pompe Disease and Prader-Willi syndrome (Ren et al., 2003; ElMallah et al., 2015). Another situation whereby obstructive and central apnea occurs is with the loss of normal excitatory drive in apnea of prematurity (Ren et al., 2015). Yet another occurs in certain neurodegenerative and cardiovascular diseases where neurons that synapse on to preBötC neurons function abnormally or die off (e.g. stroke, heart failure, brain trauma, Parkinson's Disease; reviewed in Cheng et al., 2017; Culebras and Anwar 2018).

Two models of suppressed excitatory drive to preBötC neurons and respiratory motoneurons were used to demonstrate that α4β2 nAChR targeting drugs alleviate respiratory depression associated with that general pathology. The first model was the newborn rat medullary slice preparation which was bathed in a solution containing lower extracellular potassium levels (6 mM) than normal (9 mM). This caused a lack of normal excitation and thus reduced respiratory rhythm and motor output (small amplitude and short bursting duration) on XII motoneurons. As shown in FIG. 7, the addition of A85380 (25 nM) to the bathing medium potently reversed the loss of respiratory drive and motor output.

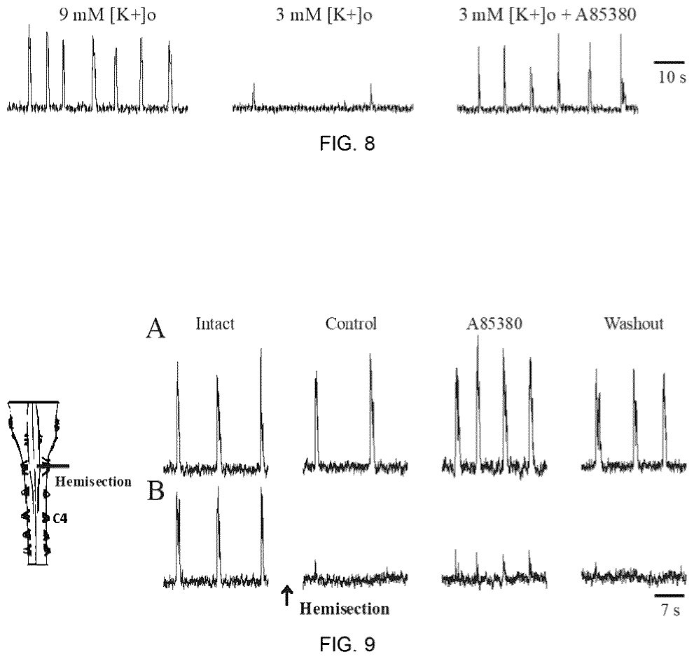

The second model was the perinatal rat brainstem spinal cord preparation which generated unstable respiratory rhythmogenesis and reduced drive to XII motoneurons due to lack of excitatory drive. Many premature infants demonstrate obstructive sleep apnea and central sleep apnea as a result of this loss of synaptic drive. As shown in FIG. 8, rectified and integrated XII nerve recordings from brainstem-spinal cord dissected at C2 shows an initial strong respiratory activity within 40 minutes, followed by period of slow respiratory frequency and weak motor output (small amplitude and short bursting duration). Bath application of A85380 (25 nM) increased respiratory frequency, amplitude and bursting duration, thereby alleviating the weak respiratory rhythmogenesis and activation of hypoglossal nerve (XIIn) motoneuron activity.

There are cholinergic projections from mespontine cholinergic neurons to the XII motoneurons and respiratory nuclei which are modulated during sleep (Kubin and Fenik, 2004; Bellingham and Ireland, 2002). Collectively, the above data provide evidence that targeting α4β2 nAChRs may alleviate multiple disorders caused by reduced excitation of the preBötC (related to central sleep apnea) and XII motoneurons (related to obstructive sleep apnea). Without being bound by any theory, activation of α4β2 nAChRs during sleep may provide sufficient excitatory input to respiratory rhythmogenic neurons and XII motoneurons to overcome any suppression related to decreased endogenous release of Ach that leads to central sleep apnea and obstructive sleep apnea, respectively.

### Example 6—Efficacy of α4β2 nAChR Targeting Drug (A85380) to Alleviate Respiratory Depression Caused by Spinal Cord Injury

One of the primary causes of morbidity and mortality associated with spinal cord injury is loss of normal functioning of the respiratory musculature. This is caused by a reduced synaptic drive from respiratory rhythm generating centers onto respiratory spinal motoneurons. An in vitro newborn brainstem spinal cord rat model of spinal cord injury (Zimmer and Goshgarian, 2005) was used to test the efficacy of targeting α4β2 nAChRs to alleviate weak respiratory drive. FIG. 9 shows a diagram of a brainstem-spinal cord preparation with C2 hemisection (left). Rectified and integrated signals were made from both C4 recordings from a P0 rat brainstem-spinal cord preparation showing the respiratory activity both contralateral (A: top traces) and ipsilateral (B: bottom traces) to one side C2 hemisection. There was a marked reduction in neural activity after the hemisection in ipsilateral nerve, but respiratory-like activity was still observed occasionally and was correlated with the respiratory activity on the contralateral nerve. Bath application of A85380 (25 nM) caused an increased in the previously weak respiratory motor output (i.e., increased the respiratory frequency) and partially increased the ipsilateral respiratory amplitude on the damaged side of the spinal cord.

Based on these data, A85380 administration may enhance the weak synaptic drive to respiratory and other motoneurons (e.g., postural, locomotor) that occurs after loss of descending drive after spinal cord injury. Without being bound by any theory, a drug therapy such as administration of A85380 to enhance respiratory activity used in conjunction with intermittent hypoxia may yield further strengthening respiratory output (Turner et al., 2016).

### Example 7—Alleviation of Fentanyl-Induced Respiratory Depression by Selective Nicotinic α4β2 Receptor Ligand VMY-2-95

The efficacy of selective α4β2 nAChR partial agonists, sazetidine-A and its analogue VMY-2-95, were examined for overcoming OIRD. α4β2 nAChR assembles into two distinct stoichiometries: (α4)₂(β2)₃ and (α4)₃(β2)₂, which are referred to as high-sensitivity (HS) and low-sensitivity (LS) nAChRs, respectively (Moroni et al., 2006). High-affinity α4β2 nAChR partial agonists, sazetidine-A and VMY-2-95, activate or potently desensitize α4β2 nAChRs (Xiao et al., 2006; Carbone et al., 2009; Levin et al., 2010; Yenugonda et al., 2013). Specifically, sazetidine-A showed full agonist activity at the (α4)₂(β2)₃ receptors but nearly no agonist activity at the (α4)₃(β2)₂ receptors (Zwart et al., 2008; Carbone et al., 2009). Compared with sazetidine-A (Hussmann et al., 2012), VMY-2-95 has a better penetration of the blood-brain barrier with oral administration (Kong et al. 2015). The present studies tested the hypothesis that sazetidine-A and VMY-2-95 alleviate fentanyl-induced respiratory depression without compromising the fentanyl-induced analgesia.

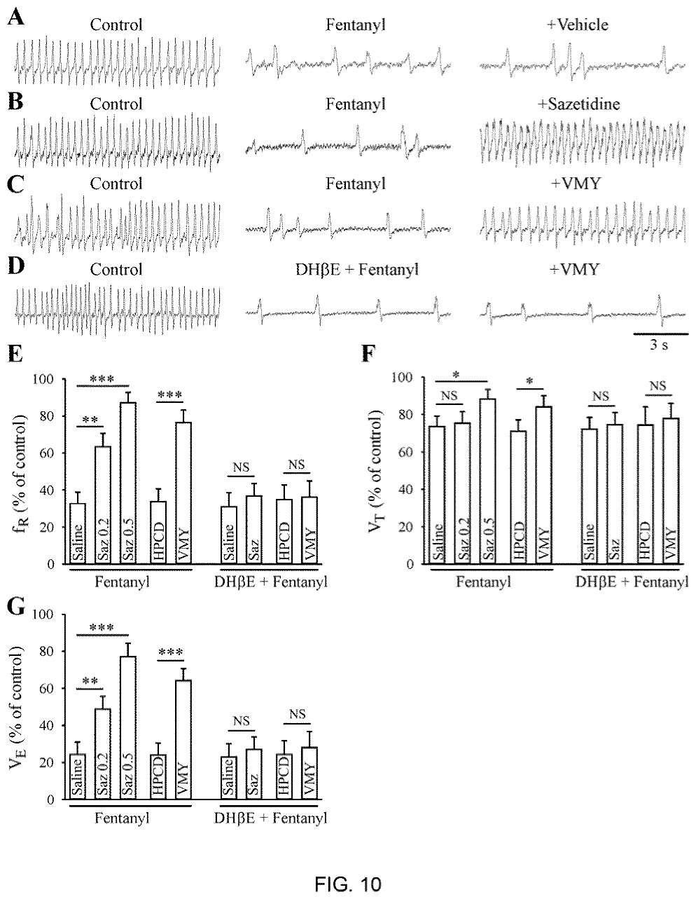

In the first study, sazetidine-A and its analogue VMY-2-95 were examined in vivo (all drugs administered via neck sc) in rat pups (postnatal day 3). Neither sazetidine-A (0.1-0.5 mg/kg), VMY-2-95 (1 mg/kg), nor α4β2/α4β4 nAChR antagonist DHβE (6 mg/kg) significantly altered baseline V_{E} (data not shown). Consistent with our previous studies (Ren et al., 2015), fentanyl (35 μg/kg) induced a marked decrease in f_{R}, and minor decrease in V_{T}, and the effects lasted for ˜20 min. The results are shown in FIG. 10. In particular, FIGS. 10A-D are representative whole body plethysmographic recordings from four postnatal day 3 pups. All drugs tested were administered subcutaneously in the posterior neck region. Administration of fentanyl (35 μg/kg, FIGS. 10A-C) or co-administration with DHβE (6 mg/kg, FIG. 10D) caused a marked depression of respiratory frequency and a mild depression of tidal volume within 7 min post fentanyl administration. Subsequent administration of vehicle (HPCD) had no effect on fentanyl-induced respiratory depression (FIG. 10A). Sazetidine-A (0.5 mg/kg, FIG. 10B) and VMY-2-95 (1 mg/kg, FIG. 10C) reversed fentanyl-induced respiratory depression. However, neither sazetidine-A (0.5 mg/kg, data not shown) nor VMY-2-95 (1 mg/kg, FIG. 10D) had any effect on respiratory depression induced by fentanyl co-administrated with DHβE (6 mg/kg). Respiratory variables were presented and measured before fentanyl administration (left traces), 7 min after fentanyl (middle traces), and 12 min after fentanyl (right traces). Population data showing respiratory frequency (f_{R}), tidal volume (V_{T}), and minute ventilation (V_{E}) relative to control prior to fentanyl administration is shown in FIGS. 10E-G. *p<0.05, **p<0.01, ***P<0.001, significant difference; ns: p>0.05, no significant difference in compared groups, using two-way repeated-measures ANOVA (Holm-Sidak methods). n=6-7 each.

In summary, the first study showed that at ˜7 min post-fentanyl, subsequent administration of vehicle (FIG. 10A) had no effect on respiratory depression. Sazetidine-A (0.2-0.5 mg/kg, FIG. 10B) and VMY-2-95 (1 mg/kg, FIG. 10C) alleviated fentanyl-induced respiratory depression (decrease in f_{R}, V_{T}, V_{E}). Co-application of DHβE (6 mg/kg) with fentanyl prevented the alleviation of fentanyl-induced respiratory depression by subsequent administration of sazetidine-A (data not shown) or VMY-2-95 (FIG. 10D).

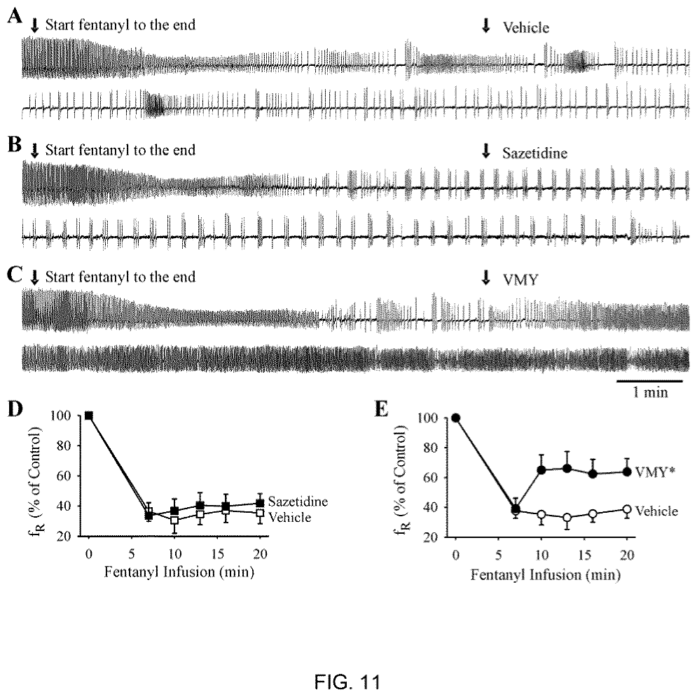

In the second study, the efficacy of α4β2 ligands sazetidine-A and its analogue VMY-2-95 to counter fentanyl-induced respiratory depression in adult rats in vivo was examined. FIG. 11 shows representative plethysmographic recordings of adult rats breathing during a 20 min iv infusion of 60 μg/kg fentanyl. Administration of fentanyl (60 μg/kg over 20 min, iv infusion) caused a marked decrease of respiratory rate in two adult rats. Traces are displayed continuously with whole-body plethysmograph recordings. FIG. 11A shows administration of vehicle (HPCD, iv bolus, approximately 7 min after fentanyl) had no effect on fentanyl-induced respiratory depression. FIG. 11B shows administration of Sazetidine-A (1 mg/kg, iv bolus, approximately 7 min after fentanyl) had no effect on fentanyl-induced respiratory depression. Finally, FIG. 11C shows administration of VMY-2-95 (1 mg/kg, iv bolus) reversed the fentanyl-induced decrease of respiratory rate. FIG. 11D is population data showing sazetidine-A (0.5-2 mg/kg) had no effects on fentanyl-induced decrease in respiratory rate (relative to control prior to fentanyl infusion). ^{NS}p>0.05, no significant difference, compared with vehicle (saline) group, using two-way repeated measures of ANOVA (followed by Holm-Sidak methods). n=8 each. FIG. 11E is population data showing of VMY (1 mg/kg)-induced alleviation of fentanyl-induced decrease in respiratory rate (relative to control prior to fentanyl infusion).

In summary, the second study showed that, similar to past studies (Ren et al., 2015), this paradigm caused a marked suppression of f_{R} (>50% decrease) in most rats within 7 min after fentanyl administration. Subsequent injection of vehicle (FIG. 11A) did not change the course of fentanyl action. Subsequent injection of sazetidine-A (0.5-2 mg/kg, FIG. 11B) did not change the course of fentanyl action. In contrast, subsequent injection of VMY-2-95 (1 mg/kg, iv, FIG. 11C) reversed the fentanyl-induced f_{R} decrease and the reversal persisted beyond the duration of the fentanyl infusion.

Fentanyl also causes a mild decrease in tidal volume (V_{T}). It was determined in past studies (Ren et al., 2006) that the reduced V_{T} is in part due to decreased drive to respiratory motoneurons and a larger component results from fentanyl-induced muscle rigidity and ribcage stiffness. The rigidity that occurs in rats (even those that do not demonstrate significant opioid-induced f_{R} decrease) is a well-documented phenomenon whose mechanism of action is poorly understood. VMY-2-95 did not reduce the muscle rigidity and thus there is not a reversal of V_{T} in rats. Note: VMY-2-95 alleviated fentanyl-induced V_{T} in pups, which typically do not show marked stiffness after fentanyl (FIGS. 10F-G).

In view of the first and second studies, the following observations were made: (1) Sazetidine-A at doses of 0.2-0.5 mg/kg reversed the fentanyl-induced respiratory depression in pups (minimal blood-brain barrier), but had no effect on fentanyl-induced respiratory depression at 0.5-2 mg/kg for adult rats; (2) VMY-2-95 at the dose of 1 mg/kg reversed fentanyl-induced respiratory depression in frequency in both pups and adult rats; (3) The differential efficacy of these two drugs on fentanyl-induced respiratory depression could be explained by a better penetration of VMY-2-95 into the blood-brain barrier than that of sazetidine-A (Hussmann et al., 2012; Kong et al. 2015); and (4) Co-application of selective α4β2/α4β4 nAChR antagonist DHβE with fentanyl prevented the alleviation of fentanyl-induced respiratory depression by subsequent administration of sazetidine-A or VMY-2-95, indicating that the alleviation of fentanyl-induced respiratory depression is via the activation, not desensitization of α4β2 nAChR.

### Example 8—Effects of SIB 1553A on Fentanyl-Induced Respiratory Depression in Newborn (Postnatal Day 3-4) Rats

As previously mentioned, the β2 and the β4 nicotinic acetylcholine receptor (nAChR) subunits are expressed throughout the central nervous system and the peripheral nervous system. These two β subunits can form heteromultimeric channels with any of the α2, α3, α4, or α5 subunits. α4β2 are among the most abundant in the mammalian brain, whereas α3β4 primarily in peripheral ganglia. The α4β2 nAChRs have been examined in pre-clinical and clinical trials of non-respiratory related functions (e.g. analgesia, cognition, smoking cessation). The above Examples show that activating α4β2 nAChRs with full agonists, partial agonists and positive allosteric modulators alleviate opioid-induced respiratory depression without decreasing opioid analgesia.

Also studied was the ability of the compound SIB-1533A, a partial agonist of α4β2 nAChRs, to counteract OIRD was examined. SIB-1533A was found it to be effective but the actions were not completely blocked by administration of a α4β²/_{α}4β4 nAChR antagonist as was the case for agents such as A85380. Thus, other mechanisms of action were considered. A further literature search revealed that SIB-1533A was also a potent agonist of β4 containing nAChRs (Menzaghi et al., 1998; Vernier et al., 1999). β4 nAChRs are involved in anxiety- and depression-like behaviors and contribute to the analgesic effects of nicotine (Semenova et al., 2012). The β4 subunit distributed in multiple areas of the rat central nervous system is a candidate assembly partner for the α4 subunit (Dineley-Miller & Patrick, 1992).

Thus, the effects of SIB 1553A on fentanyl-induced respiratory depression in newborn (postnatal day 3-4) rats were studied. FIGS. 12A and B are representative whole body plethysmographic recordings from two pups. All drugs tested were administered subcutaneously in the posterior neck region. FIG. 12A shows that the administration of fentanyl (35 μg/kg) co-administered with saline vehicle caused a marked depression of respiratory frequency and a mild depression of tidal volume within 7 min post fentanyl administration. Subsequent administration of SIB 1553A (40 mg/kg) partially reversed fentanyl-induced decrease in respiratory rate (f_{R}), without marked effects on fentanyl-induced decrease in tidal volume (V_{T}). FIG. 12B shows that the administration of fentanyl (35 μg/kg) co-administered with non-selective nAChR receptor antagonist mecamylamine (6 mg/kg) did not affect the fentanyl-induced respiratory depression. Subsequent administration of SIB 1553A (40 mg/kg, 7 min post fentanyl) had no effects on fentanyl-induced respiratory depression. Respiratory variables were presented and measured before fentanyl administration (left traces), 7 min after fentanyl (middle traces), and 12 min after fentanyl (right traces). FIG. 12C shows population data showing f_{R} relative to control prior to fentanyl administration. *p<0.05, **p<0.01, ***p<0.001, significant difference; ^{NS}p>0.05, no significant difference in compared groups, using two-way repeated-measures ANOVA (Holm-Sidak method). n=5-7 each.

Consistent with previous studies (Ren et al., 2015), fentanyl (35 μg/kg) induced a marked decrease in f_{R}, and minor decrease in V_{T}, and the effects lasted for ˜20 min. At ˜7 min post-fentanyl, subsequent administration of SIB 1553A (40 mg/kg, FIG. 12A) alleviated fentanyl-induced decrease in f_{R}, without much effects on V_{T}. The SIB 1553A effects were abolished by pre-administration of non-selective nAChR antagonist mecamylamine (6 mg/kg, neck sc, co-administration with fentanyl, FIG. 12B), but only partially blocked by selective α4β2 and α4β4 nAChRs antagonist DHβE (6 mg/kg, co-administration with fentanyl). It should be noted that DHβE blocks the activation of α4β2, α4β4 nAChRs, with IC50 of 0.37, 0.19 μM, respectively (Harvey et al., 1996, Table 1).

### Example 9—Alleviation of Fentanyl-Induced Respiratory Depression by Nicotinic Acetylcholine Receptor Agonist Lobeline and Role of Beta4 Containing nAChRs

Given the results with SIB-1533A, the potential involvement of β4-containing nAChRs was explored using additional pharmacological probes. Lobeline is a full agonist at α4β4 human nAChR, partial agonist at α4β2 human nAChR, partial agonist at α3β4 rat nAChR (Wu et al., 2006; Kaniakova et al., 2014). Lobeline may also function as a μ-opioid receptor antagonist at tenfold of doses of interacting with nAChR (Miller et al., 2007). Lobeline has been widely used in smoking remedies, without severe adverse effects. Lobeline induces analgesia, enhances nicotine-induced analgesia in mice, via mecamylamine-sensitive nAChR, but not via α4β2 (Damaj et al., 1997). Thus, it was investigated whether lobeline alleviated the fentanyl-induced respiratory depression without compromising the fentanyl-induced analgesia.

(i) Countering Fentanyl-Induced Respiratory Depression in Neonatal Rats by Lobeline Partially Via Activation of β4 nAChRs

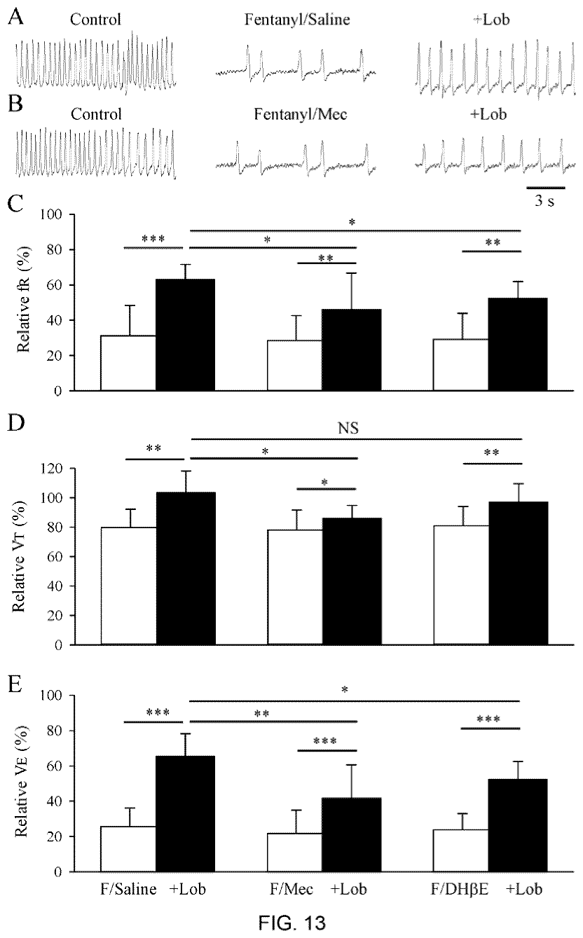

nAChRs targeting agents in vivo (all drugs administered via neck sc) were studied in rat pups. Representative whole body plethysmographic recordings from two pups are shown in FIGS. 13A and B. All drugs tested were administered subcutaneously in the posterior neck region. FIG. 13A showed administration of fentanyl (35 μg/kg) co-administered with saline vehicle caused a marked depression of respiratory frequency and a mild depression of tidal volume within 7 min post fentanyl administration. Subsequent administration of lobeline (10 mg/kg) partially reversed fentanyl-induced decrease in respiratory rate (f_{R}), and completely reversed fentanyl-induced decrease in tidal volume (V_{T}). FIG. 13B showed that administration of fentanyl (35 μg/kg) co-administered with non-selective nAChR receptor antagonist mecamylamine (6 mg/kg) did not affect the fentanyl-induced respiratory depression. Subsequent administration of lobeline (10 mg/kg, 7 min post fentanyl) had less effects on fentanyl-induced respiratory depression. Respiratory variables were presented and measured before fentanyl administration (left traces), 7 min after fentanyl (middle traces), and 12 min after fentanyl (right traces). Population data showing f_{R}, V_{T}, and minute ventilation (V_{E}) relative to control prior to fentanyl administration is shown in FIGS. 13C-E. *p<0.05, **p<0.01, ***p<0.001, significant difference; ^{NS}p>0.05, no significant difference in compared groups, using two-way repeated-measures ANOVA (Holm-Sidak method). n=7-8 each.

Consistent with previous studies (Ren et al., 2015), fentanyl (35 μg/kg) induced a marked decrease in f_{R}, and minor decrease in V_{T}, and the effects lasted for ˜20 min. At ˜7 min post-fentanyl, subsequent administration of lobeline (10 mg/kg, FIG. 13A) alleviated fentanyl-induced respiratory depression (decrease in f_{R}, V_{T}, V_{E}). The lobeline effects on fentanyl-induced respiratory depression were markedly suppressed, but not completely abolished, by pre-administration of non-selective nAChR antagonist mecamylamine (6 mg/kg, neck sc, co-administration with fentanyl, FIG. 13B). The lobeline effects on fentanyl-induced respiratory depression in f_{R} (but not V_{T}) were partially suppressed by selective α4β4/α4β2 nAChR antagonist DHβE (6 mg/kg, neck sc, co-administration with fentanyl). Population data was shown in FIGS. 13C-E. It should be noted that it was previously demonstrated that activation of α7 nAChRs had no effects on fentanyl-induced respiratory depression. These results suggested: (1) the effects of lobeline on fentanyl-induced respiratory depression in f_{R} were partially mediated by α4β4 (α4β2 might as well) nAChRs; and (2) the effects of lobeline on fentanyl-induced decrease in V_{T} were mediated by β4 (e.g. α3β4, α4β4/α4β2) nAChRs. Overall, β4 was involved in the effects of lobeline on fentanyl-induced respiratory depression.

(ii) Countering Fentanyl-Induced Respiratory Depression in Adult Rats by Lobeline

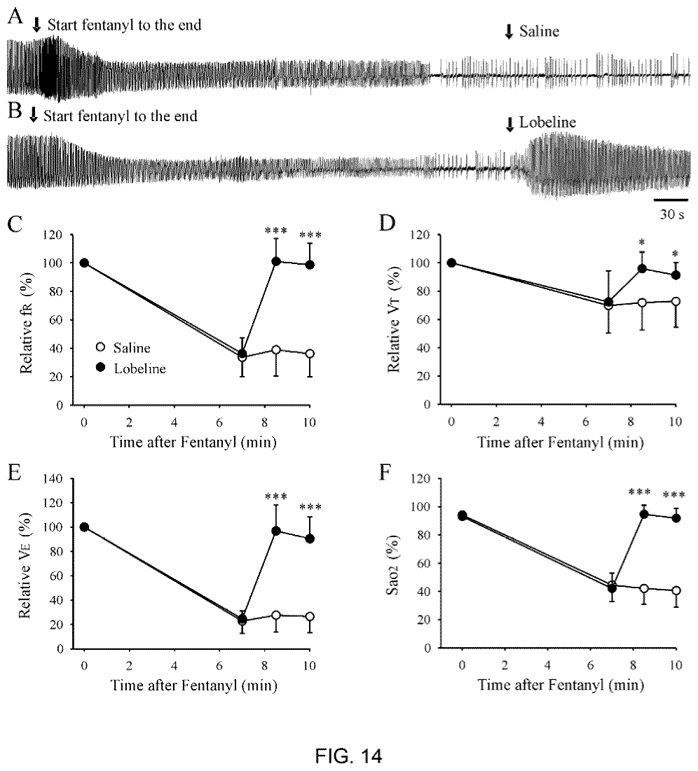

The efficacy of lobeline to counter fentanyl-induced respiratory depression in adult rats in vivo was then examined. As shown in FIGS. 14A and B, administration of fentanyl (30 μg/kg over 10 min, iv infusion) caused a marked respiratory depression (f_{R}, V_{T}, and V_{E}) in whole body plethysmographic recordings from two rats. FIG. 14A showed that subsequent administration of saline vehicle (iv) had no effect on fentanyl-induced respiratory depression. FIG. 14B showed that subsequent administration of lobeline (3 mg/kg, iv bolus, approximately 7 min after fentanyl) completely reversed fentanyl-induced respiratory depression. FIGS. 14C-E shows population data showing respiratory parameters (f_{R}, V_{T}, V_{E}) relative to control prior to fentanyl infusion. n=9 each. FIG. 14F shows population data showing arterial oxygen saturation (Sao₂). n=6 each. *p<0.01, ***p<0.001, significant difference in two groups, using two-way repeated measures of ANOVA (followed by Holm-Sidak method).

In summary, FIG. 14 shows representative plethysmographic recordings of adult rats breathing during a 10 min iv infusion of 30 μg/kg fentanyl. Similar to past studies (Ren et al., 2009, 2015), this paradigm caused a marked respiratory depression (decrease in f_{R}, V_{T}, and V_{E}) in most rats within 7 min after fentanyl administration. Subsequent injection of saline vehicle (FIG. 14A) did not change the course of fentanyl action. In contrast, subsequent injection of lobeline (3 mg/kg, FIG. 14B) completely reversed the fentanyl-induced respiratory depression in f_{R}, V_{T}, V_{E}, and Sao₂ (population data shown in FIGS. 14C-F).

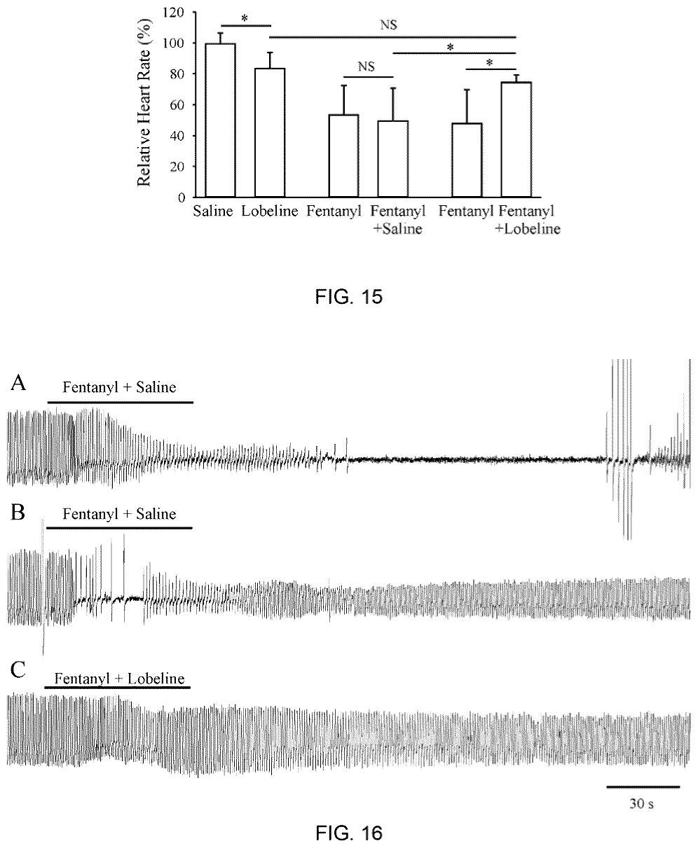

It should be noted that (1) it appears that the fentanyl-induced intense muscle stiffness observed in most rats may have been mildly alleviated, but still persisted in the presence of lobeline and (2) administration of lobeline (3 mg/kg, iv) alone caused a mild increase in V_{E} (116.2±8.7% of control, vs. saline 105.2±4.7% of control, p=0.038, n=5 each), consistent with a previous study (Sloan et al., 1988). Administration of lobeline (6 mg/kg, ip, n=3) alone did not affect V_{E}. 3) As shown in FIG. 15, administration of lobeline (3 mg/kg, iv) alone caused mild bradycardia; consistent with a previous study (Sloan et al., 1988). In FIG. 15, data point shown 2 min post saline (iv, n=5), 2 min post lobeline (3 mg/kg, iv, n=5), 7 min post fentanyl (30 μg/kg over 10 min iv infusion), and 2 min after subsequent administration of saline (n=8) or lobeline (n=8). *p<0.05, showed significant difference; ^{NS}p>0.05, no significant difference. T-test was used for comparison of saline-treated and lobeline-treated groups; Kruskal-Wallis one way ANOVA on ranks (Tukey Test) was used for comparison of fentanyl and subsequent saline or lobeline-treated groups. Administration of fentanyl (30 μg/kg over 10 min iv infusion) causing severe bradycardia, subsequent administration of saline did not change the severity of bradycardia. Interestingly, subsequent administration of lobeline did not exaggerate bradycardia, instead markedly alleviated fentanyl-induced bradycardia.

(iii) Prevention of Fentanyl-Induced Respiratory Depression and Apnea in Adult Rats by Lobeline

Fentanyl induces a particularly strong respiratory depression and apneas when administrated rapidly. The potency of lobeline on preventing respiratory depression and apneas induced by a quick administration of fentanyl was further examined.

FIGS. 16A and B show that co-administration of fentanyl (12 μg/kg over 1 min, iv infusion) with saline vehicle (1 min iv infusion) caused a marked respiratory depression (f_{R}, V_{T}, and V_{E}) and apneas in two rats. FIG. 16C showed co-administration of fentanyl (12 μg/kg over 1 min, iv infusion) with lobeline (3 mg/kg over 1 min, iv infusion) markedly prevented fentanyl-induced respiratory depression (f_{R}, V_{T}, and V_{E}) and abolished apneas.

In summary, co-administration of fentanyl (12 μg/kg over 1 min, iv) and saline (second tail vein, iv) induced marked respiratory depression. In some animals, the rate and depth of breathing was gradually suppressed by fentanyl, followed by marked apneas (FIG. 16A, n=2). In other animals, apneas appeared shortly (approximately 10-30 sec) after a quick administration of fentanyl, followed by shallow breathing (FIG. 16B, n=2). In contrast, co-administration of fentanyl (12 μg/kg over 1 min, iv) and lobeline (3 mg/kg over 1 min, second tail vein, iv, n=4) completely prevented fentanyl-induced respiratory depression and blocked apneas.

(iv) Fentanyl-Induced Analgesia in Adult Rats Unaffected or Enhanced by Lobeline

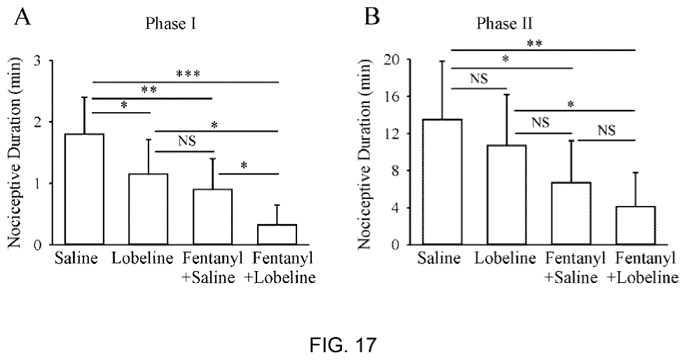

A formalin test was performed that scored time spent engaging in nociceptive behaviors (licking plus lifting of injured paw) 0-5 minutes (phase I), 20-40 minutes (phase II) post formalin injection. Formalin was administrated 30 min post lobeline, or saline. First, the effects of lobeline (3 mg/kg, iv) on baseline nociception in adult rats was assessed (FIG. 17). Formalin (1.5%, 50 μl) was injected into the intraplantar region of hind paw, at 30 min after lobeline (3 mg/kg, tail iv) or saline W/O fentanyl (10 μg/kg over 10 min iv infusion). Effects of lobeline on the time spent engaging in nociceptive behaviors (licking and lifting) were measured in phase I: 0-5 min, and phase II: 20-40 minutes post formalin. *p<0.05, **p<0.01, ***p<0.001, significant difference; ^{NS}p>0.05, no significant difference in compared groups, using one way ANOVA (Holm-Sidak method). n=8-9 each.

The lobeline-treated group had a decreased nocifensive response in the phase I relative to the saline group (FIG. 17A). The lobeline treatment tended to decrease the nocifensive response in the phase II, but the difference is not significant relative to the saline group (FIG. 17B). Then, the effects of lobeline (3 mg/kg, 7 min post fentanyl, FIG. 17) on fentanyl (30 μg/kg over 10 min iv infusion)-induced analgesia in adult rats was assessed. Fentanyl administration, with subsequent administration of saline 7 min post fentanyl, induced marked analgesia as measured by the formalin tests, whereas subsequent administration of lobeline further increased fentanyl-induced analgesia in the phase I (FIG. 17A). Lobeline tended to increase the fentanyl-induced analgesia in the phase II, but the difference is not significant relative to saline group (FIG. 17B). Lobeline had no effects on fentanyl (30 μg/kg over 10 min iv infusion)-induced sedation (loss of righting reflex: 22.4±7.3 min vs. vehicle: 23.8±4.6 min, p=0.64).

In summary, lobeline, and potentially other agonists or modulators of (34 nAChRs, can reduce OIRD and thus has the potential for advancing pain control and reducing opioid-induced respiratory depression and overdose.

### Example 10—Potentiation of Baseline Respiratory Rhythm and Alleviation of DAMGO-Induced Respiratory Depression in In Vitro Via Activation of α4β2 nAChRs

Neonatal (postnatal day 1-3) rats were anesthetized with metofane, decerebrated and the brainstem-spinal cord dissected as previously reported. The neuraxis was continuously perfused at 27°±1° C. (5 ml/min; chamber volume, 3 ml) with modified Kreb's solution that contained 128 mM NaCl, 3.0 mM KCl, 1.5 mM CaCl₂, 1.0 mM MgCl₂, 23.5 mM NaHCO₃, 0.5 mM NaH₂PO₄, and 30 mM d-glucose equilibrated with 95% O₂-5% CO₂ (pH 7.4).

A single transverse slice containing the preBötC and more caudal reticular formation regions was then cut (700 μm thick) from brainstem-spinal cord preparations perfused with a bathing solution identical to that used for B SSC preparation with the exception that the KCl concentration was increased to 9 mM to facilitate long-term generation of stable rhythm. Recordings from the fourth ventral cervical nerve roots of brainstem-spinal cord or hypoglossal nerve roots of medullary slice preparations were amplified, rectified, low-pass filtered, and recorded to a computer, using an analog-digital converter (Axon Instruments Digidata; Molecular Devices, Sunnyvale, Calif.) and data acquisition software (Axon Instruments AxoScope).

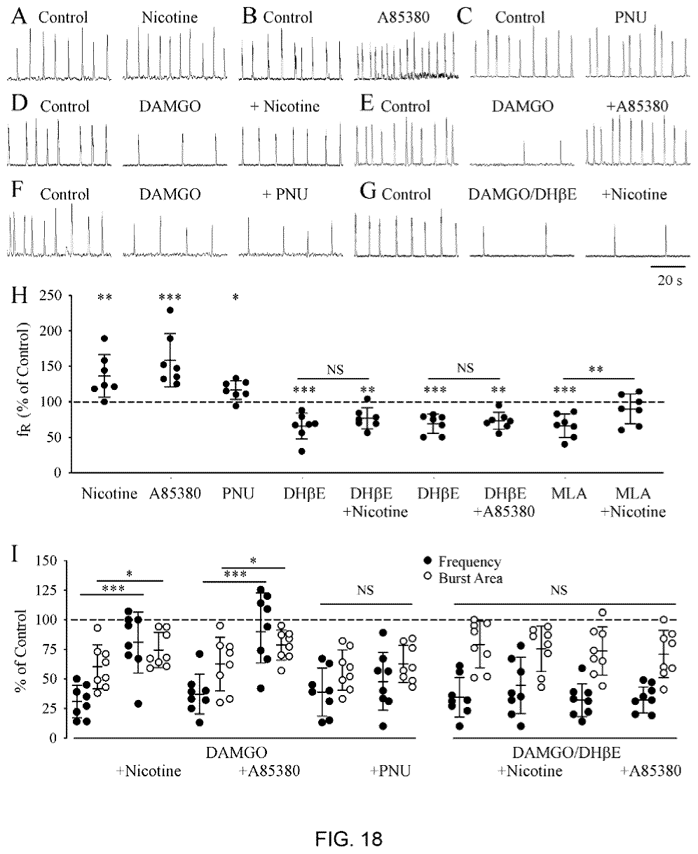

The in vitro analyses were commenced by examining the effects of drug application to media bathing brainstem-spinal cord preparations that generate spontaneous inspiratory motor activity (FIG. 18). Nicotine (600 nM, FIG. 18A), a non-selective agonist of nAChR, caused an increase of baseline f_{R}, at concentrations above 200 nM. The selective α4β2 nAChR agonist A85380 (25 nM, FIG. 18B) increased baseline f_{R} at concentrations above 5 nM, whereas activation of the α7 nAChR with the agonist PNU282987 (30 μM, FIG. 18C) resulted in a very modest increase in f_{R}. The μ-opiate receptor agonist, D-Ala2, N-Me-Phe4, Gly5-ol]enkephalin or DAMGO (200 nM), was used to induce respiratory depression in vitro. There was a clear reversal of DAMGO-induced respiratory depression (decrease in f_{R} and burst area) by subsequent application of nicotine (600 nM, FIG. 18D) or A85380 (25 nM, FIG. 18E), but not by PNU282987 (30 μM, FIG. 18F). The effects of nicotine and A85380 on baseline and DAMGO-induced respiratory depression were blocked by pre-application of the α4β2 nAChR antagonist DHβE (400 nM, FIGS. 18G & 18H), but not by the α7 nAChR antagonist MLA (400 nM, FIG. 18H). Consistent with a previous study, f_{R} was slower in the presence of DHβE (400 nM, FIG. 18H) or MLA (400 nM, FIG. 18H), indicating tonic excitation of respiratory rhythm by endogenous activation of α4β2 and α7 nAChRs. The above nAChR targeting agents had no significant effects on inspiratory amplitude, duration, or area (data not shown).

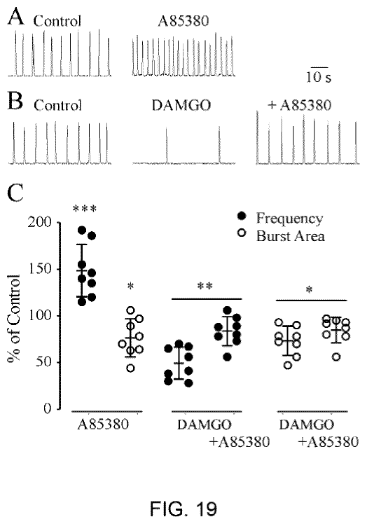

Next, the medullary slice preparation (FIG. 19) that contains the preBötC and a population of XII motoneurons that discharge during the inspiratory phase of the respiratory cycle were utilized. This allows for a more direct assessment of drug action at the level of the preBötC. Bath application of A85380 (25 nM) increased f_{R}, whereas PNU282987 (30 μM, data not shown) had no effect on baseline respiratory activity. DAMGO (200 nM) caused a suppression of f_{R} that was alleviated by A85380 (25 nM), but not by PNU282987 (30 μM, data not shown).

### Example 11—Countering Fentanyl-Induced Respiratory Depression in In Vivo Neonatal Rats by Activation of α4β2 nAChRs

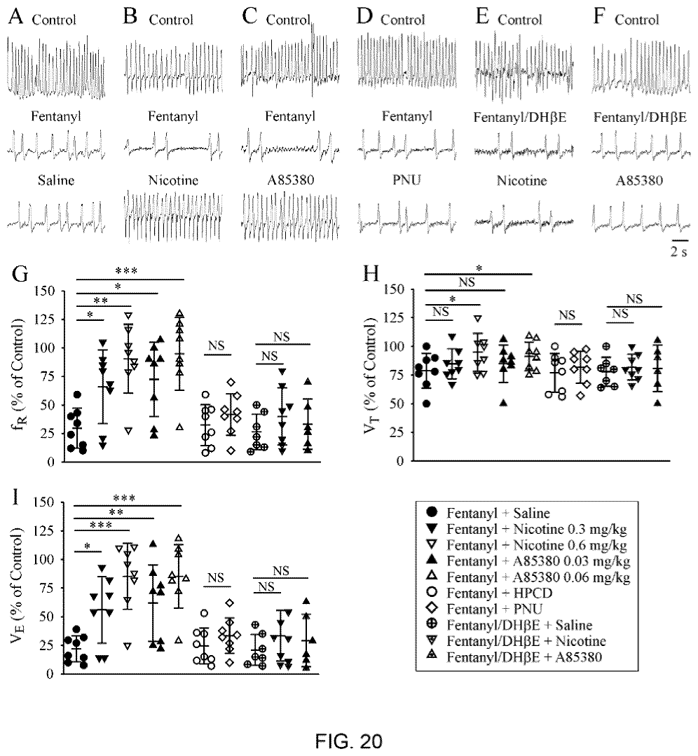

The compounds in Example 10 were then tested in vivo (all drugs administered via neck sc) in rat pups of similar age as those used in vitro. Neither nicotine (0.3-0.6 mg/kg), A85380 (0.03-0.06 mg/kg), PNU282987 (1-20 mg/kg) nor DHβE (6 mg/kg) significantly altered baseline V_{E} (data not shown). Consistent with previous studies, fentanyl (35 μg/kg) induced a marked decrease in f_{R}, and minor decrease in V_{T}, and the effects lasted for ˜20 min. At ˜7 min post-fentanyl, subsequent administration of vehicle (saline: FIG. 20A or HPCD) had no effect on respiratory depression. Nicotine (0.3-0.6 mg/kg, FIG. 20B) and A85380 (0.03-0.06 mg/kg, FIG. 20C) alleviated fentanyl-induced respiratory depression (decrease in f_{R}, V_{T}, V_{E}) in a dose-dependent manner, whereas there was no reversal by PNU282987 (1-20 mg/kg, FIG. 20D). Co-application of DHβE (6 mg/kg) with fentanyl prevented the alleviation of fentanyl-induced respiratory depression by subsequent administration of nicotine (FIG. 20E) or A85380 (FIG. 20F). Population data was shown in FIGS. 20G-I, showing respiratory frequency (f_{R}), tidal volume (V_{T}), and minute ventilation (V_{E}) relative to control prior to fentanyl administration. *p<0.05, **p<0.01, ***P<0.001, significant difference; ns: p>0.05, no significant difference in compared groups, using two-way repeated-measures ANOVA (Holm-Sidak methods). n=6-8 each.

### Example 12—Countering Fentanyl-Induced Respiratory Depression in In Vivo Adult Rats by α4β2 nAChR Agonists

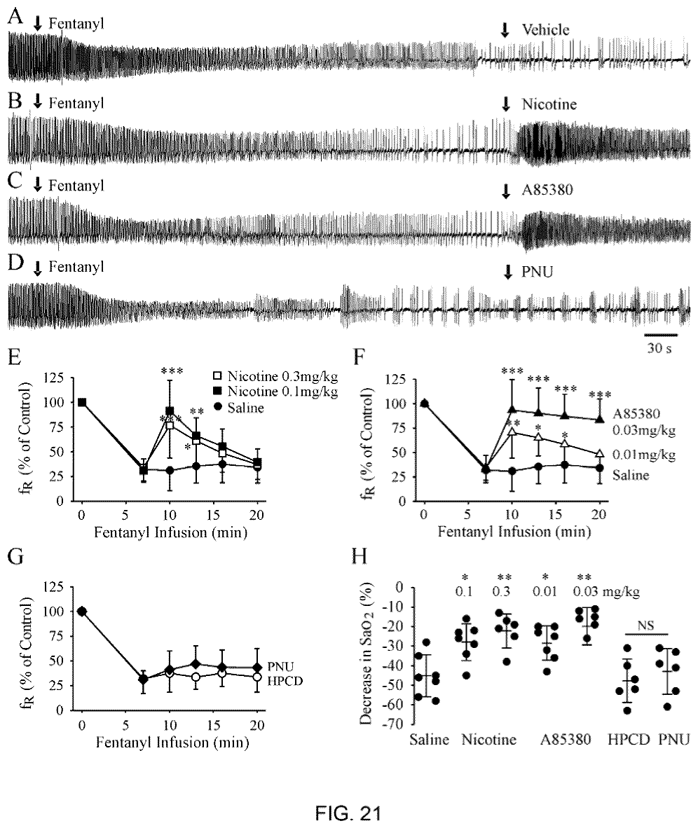

The efficacy of α4β2 and α7 nAChR agonists to counter fentanyl-induced respiratory depression in adult rats in vivo was examined. FIG. 21 shows representative plethysmographic recordings of adult rats breathing during a 20 min iv infusion of 60 μg/kg fentanyl. Similar to past studies, this paradigm caused a marked suppression of f_{R} (>50% decrease) in most rats within 7 min after fentanyl administration. Subsequent injection of vehicle (FIG. 21A) did not change the course of fentanyl action. In contrast, subsequent injection of nicotine (0.1-0.3 mg/kg, iv, FIG. 21B) reversed the fentanyl-induced f_{R} decrease and the reversal lasted 5-10 minutes. Injection of A85380 (0.01-0.03 mg/kg, iv, FIG. 21C) dose-dependently reversed the fentanyl-induced f_{R} decrease and at the highest dose the reversal persisted beyond the duration of the fentanyl infusion. PNU282987 (1-10 mg/kg, iv, FIG. 21D) had no effect on the fentanyl-induced respiratory depression. Population data was shown in FIGS. 21E-G, showing the time course of changes of respiratory frequency relative to control prior to drug administration, with nicotine (FIG. 21E) and A85380 (FIG. 21F) compared with saline, PNU282987 (1, 2, 3, 10 mg/kg, n=2 each dose) compared with HPCD (FIG. 21G). *p<0.05, **p<0.01, ***p<0.001, significant difference, using two-way repeated-measures ANOVA (Holm-Sidak methods). n=8 each. The fentanyl-induced decrease in oxygen saturation was alleviated after administration of nicotine, A85380, but not by PNU282987 (FIG. 21H). The reversal of fentanyl-induced decrease in f_{R} by A85380 (0.03 mg/kg) was rapid (median onset of effect: 11.1s, n=8) and comparable to the reversal caused by naloxone (0.3 mg/kg, 10.1s, n=4, Mann-Whitney Rank Sum test, p=0.68). Fentanyl-induced decrease of body temperature at the end of the 20-min infusion of 60 μg/kg fentanyl was not significantly different with saline (median: −1.1° C., n=6) or A85380 (median: −1.2° C., n=6, Mann-Whitney Rank Sum test, p=0.59) treatments.

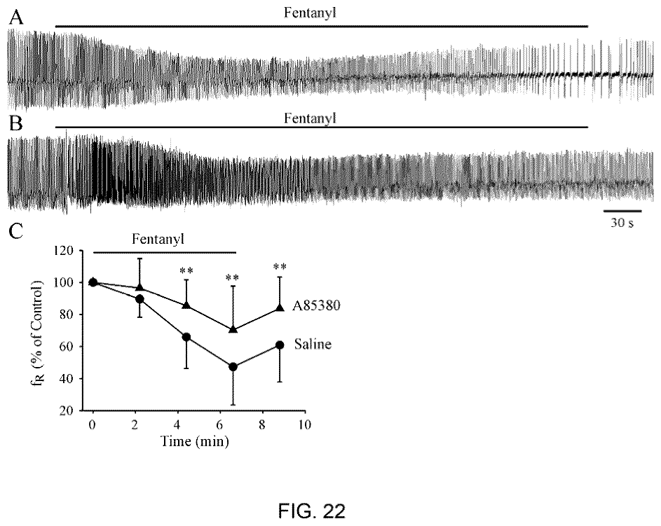

The efficacy of A85380 to prevent fentanyl-induced decrease of f_{R} was examined in adult rats. Vehicle (saline) or A85380 (0.06 mg/kg, neck sc) was injected 2 min prior to fentanyl administration (20 μg/kg, 400s infusion, FIG. 22). It caused a marked f_{R} decrease in most vehicle treated animals. Administration of A85380 reduced the severity of fentanyl-induced respiratory depression. Note that the 0.06 mg/kg dose administered is approximately the EC₅₀ based on previous rat studies of A85380. Consistent with those studies, no behavioral side effects were observed. In particular, FIG. 22A shows that 2 min after saline (neck subcutaneously), administration of fentanyl (20 μg/kg over 400s, iv infusion) caused a marked depression of respiratory frequency and minute ventilation. FIG. 22B shows that pre-administration of A85380 (0.06 mg/kg, subcutaneously) 2 min prior to fentanyl reduced the fentanyl-induced decrease of respiratory frequency. FIG. 22C is population data showing respiratory frequency relative to control prior to drug administration. **p<0.01, significant difference in two groups, using two-way repeated-measures ANOVA (Holm-Sidak methods). n=16 each.

Fentanyl also caused a mild decrease in V_{T} in vivo. Past studies have determined that the reduced tidal volume is in part due to decreased drive to respiratory motoneurons and a larger component results from fentanyl-induced muscle rigidity and ribcage stiffness. The rigidity that occurs in rats is a well-documented phenomenon, possibly involving striatal μ-opioid receptors. A85380 did not appear to reduce the muscle rigidity and thus there is no reversal of V_{T} in adult rats. In contrast in β3 pups, where fentanyl-induced muscle rigidity is much less severe, there was a reversal of the V_{T} depression after nicotine or A85380.

### Example 13—Prevention of Remifentanil-Induced Apnea in In Vivo Adult Rats by α4β2 nAChR Agonist

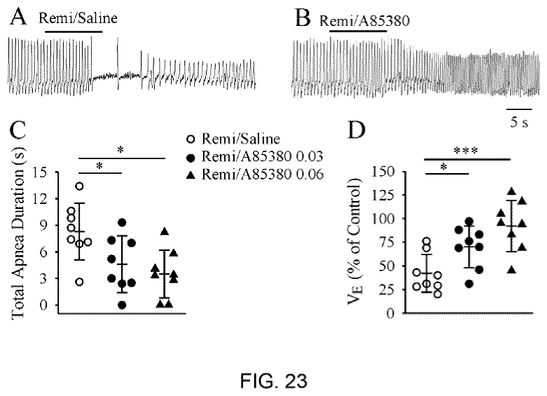

The opioid remifentanil induces a particularly strong respiratory depression that is short acting. It has proven to be more difficult to counter by either a low dose of naloxone, or an ampakine (recent clinical trial). Thus, in order to further assess the potency of A85380, it was tested against remifentanil-induced respiratory depression (FIG. 23). FIGS. 23A and B are representative whole body plethysmographic recordings from 2 adult rats. A bolus of remifentanil (5 μg/kg iv bolus over 20s, co-administrated with saline) caused marked apneas and decreased minute ventilation (V_{E}) in the first minute (FIG. 23A). Co-administration of A85380 (0.06 mg/kg, iv) with remifentanil markedly reduced the remifentanil-induced apneas and decrease in V_{E} (FIG. 23B). FIGS. 23C-D show population data. *p<0.05, ***P<0.001, significant difference in compared groups, using one way ANOVA (Holm-Sidak methods). n=8 each.

It was shown that co-administration of remifentanil (5 μg/kg over 20 sec) and saline induced marked apneas and decreased V_{E} during the first minute post-injection that recovered after 2 minutes. Co-administration of remifentanil (5 μg/kg) and A85380 (0.03-0.06 mg/kg) markedly reduced the incidence of apnea and reduced the depression of V_{E} that normally occurred immediately after remifentanil injection.

### Example 14—Fentanyl-Induced Sedation Alleviated in Adult Rats by α4β2 nAChR Agonist

In addition to respiratory depression, unintended sedation is another serious opioid-induced adverse event which contributes to patient morbidity and increased length of hospitalization. Although the underlying mechanisms are not fully understood, opioid-induced sedation is thought to involve the anticholinergic activity of opioids. Thus, the hypothesis that activation of α4β2 by A85380 (0.06 mg/kg, sc) would reduce the sedation induced by fentanyl (20 μg/kg over 400s infusion) was tested. The sedation (loss of righting reflex) was modestly, but significantly, shortened in the A85380 (16.2±1.3 min post fentanyl, n=8, two tailed t-test, p=0.039) vs vehicle treated group (20.5±1.4 min, n=8).

### Example 15—Fentanyl-Induced Analgesia in Adult Rats Enhanced by α4β2 nAChR Agonist

The effects of A85380 (0.06 mg/kg, sc) on baseline nociception in adult rats was tested. Two nociceptive tests were performed: 1) Hot plate test (Ren et al., 2006; 2015): Thermal nociception was measured with a plantar test apparatus (Ugo Basile, Comerio Va., Italy), consisting of an infrared heat source (with heat setting at 70) positioned directly beneath the hind paw, 20 mm below the chamber floor. When the rat perceived pain and withdrew its paw, the instrument automatically detected the withdrawal latency to the nearest 0.1 s. The heat stimulus was automatically terminated if a withdrawal response was not observed within 20 s of its onset to avoid the tissue damage. 2) Formalin test: A dilute solution of formalin (50 μl, 1.5% formalin) was injected into the intraplantar region of the right hind paw, followed by assessment of nocifensive behaviors (licking/lifting/flinching of the injected paw) in the second phase (20-40 minutes; reflecting inflammation) of the assay (Dubuisson and Dennis, 1977). A simple sum of time spent on licking/lifting is a recognized assessment of formalin-induced nocifensive behaviors (Abbott et al., 1995).

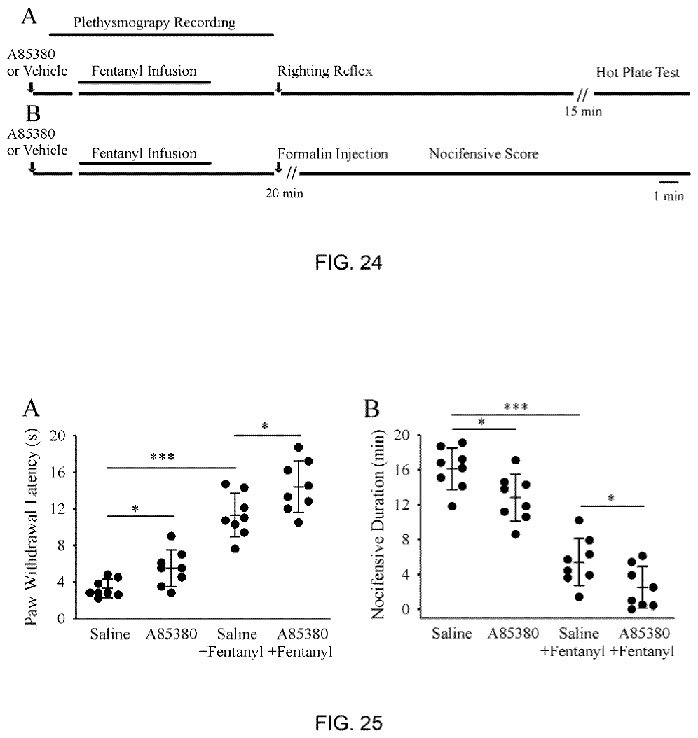

Sedation (loss of righting reflex) is defined as the rat's inability to right itself into the prone position after the animal was placed supine by repositioning the plethysmograph chamber. Animals tested in this study were unable to right when placed supine at approximately 10 min after starting fentanyl infusion, but regained a righting reflex within 30 min post-fentanyl. The onset of loss of righting reflex was not tested, therefore, duration of loss of righting reflex in this study was arbitrarily defined as the time interval from the beginning of fentanyl administration to recovery of righting reflex. FIG. 24 provides a graphic outline of the experimental protocol. A85380 (0.06 mg/kg, neck subcutaneously, sc) or saline was administrated 2 min prior to fentanyl (20 μg/kg over 400 s, iv infusion). In one set of animals, the righting reflex testing started 10 min post-fentanyl, and then the animal was removed from the chamber for thermal nociception testing 40 min post-fentanyl (FIG. 24A). In another set of animals, formalin was administered 10 min post-fentanyl (FIG. 24B).

The effects of A-85380 on paw withdrawal latency in response to thermal stimuli, measured at 42 min after A85380 or saline W/O subsequent fentanyl infusion is shown in FIG. 25A. FIG. 25B shows the effects of A85380 on the time spent engaging in nociceptive behaviors (licking and lifting) 20-40 minutes post formalin, measured at 32-52 min after A85380 or saline without subsequent fentanyl infusion. *p<0.05, **p<0.01, ***P<0.001, significant difference in compared groups, using one way ANOVA (Holm-Sidak method). n=8 each.

The paw withdrawal latency to thermal stimuli prior to and 42 min post-A85380 or saline administration showed a marked increase in paw withdrawal latency post treatment with A85380; whereas there was no change in the vehicle group. The formalin test that scored time spent engaging in nociceptive behaviors (licking plus lifting of injured paw) 20-40 minutes post formalin injection showed that the A85380-treated group had a decreased nocifensive response relative to the saline group. A85380-induced basal analgesia in both tests was consistent with a previous study. The effects of A85380 (0.06 mg/kg, 2 min prior to fentanyl) on fentanyl (20 μg/kg over 400s iv infusion)-induced analgesia in adult rats showed that fentanyl administration (pretreatment with saline) induced marked analgesia as measured by the thermal and formalin tests, whereas pretreatment of A85380 further increased fentanyl-induced analgesia in both tests. Collectively, these data indicate that fentanyl-induced analgesia was enhanced by A85380.

It should be apparent, however, to those skilled in the art that many more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

All publications mentioned in this paragraph are incorporated herein by reference (where permitted) to disclose and describe the methods and/or materials in connection with which the publications are cited. The publications in this paragraph and discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
Abbott F V, Franklin K B J, Westbrook R F (1995) The formalin test: scoring properties of the first and second phases of the pain response in rats. Pain 60 (1): 91-102.
Agro F, Salvinelli F, Casale M, Gherardi S (2004) Difficulty in airway management during sedation of patients affected by obstructive sleep apnea. Can J Anaesth 51(3):279.
Ballanyi K, Panaitescu B, Ruangkittisakul A (2010) Indirect opioid actions on inspiratory pre-Botzinger complex neurons in newborn rat brainstem slices. Adv Exp Med Biol 669:75-9.
Bannon A W, Decker M W, Holladay M W, Curzon P, Donnelly-Roberts D, Puttfarcken P S, Bitner R S, Diaz A, Dickenson A H, Porsolt R D, Williams M, Arneric S P: Broad-spectrum, non-opioid analgesic activity by selective modulation of neuronal nicotinic acetylcholine receptors. Science 1998; 279:77-81
Bellingham M C, Ireland M F (2002) Contribution of cholinergic systems to state-dependent modulation of respiratory control. Respir Physiol Neurobiol 131(1-2):135-44.
Bencherif M, Byrd G, Caldwell W S, Hayes J R, Lippiello P M (1997) Pharmacological Characterization of RJR-2403: A Nicotinic Agonist with Potential Therapeutic Benefit in the Treatment of Alzheimer's Disease. CNS Drug Reviews 3 (4):325-45.
Benyamin R, Trescot A M, Datta S, Buenaventura R, Adlaka R, Sehgal N, Glaser S E, Vallejo R (2008) Opioid complications and side effects. Pain Physician 11 (2): S105-20.
Bitner R S, Nikkel A L, Curzon P, Donnelly-Roberts D L, Puttfarcken P S, Namovic M, et al (2000) Reduced nicotinic receptor-mediated antinociception following in vivo antisense knock-down in rat. Brain Res 871:66-74.
Carbone A L, Moroni M, Groot-Kormelink P J, Bermudez I (2009) Pentameric concatenated (a4)2(b2)3 and (a4)3(b2)2 nicotinic acetylcholine receptors: subunit arrangement determines functional expression. Br J Pharmacol 156:970-981.
CCENDU Bulletin (2015) Deaths Involving Fentanyl in Canada, 2009-2014.
CDC: Drug Overdose Death Data. 2017 https://www.cdc.gov/drugoverdose/index.html
Chamberlin N L, Bocchiaro C M, Greene R W, Feldman J L (2002) Nicotinic excitation of rat hypoglossal motoneurons. Neuroscience 115:861-70.
Cheng S, Stark C D, Stark R J. (2017) Sleep apnoea and the neurologist. Pract Neurol. 17(1):21-27.
Culebras A, Anwar S. (2018) Sleep Apnea Is a Risk Factor for Stroke and Vascular Dementia. Curr Neurol Neurosci Rep. 25; 18(8):53
Curzon P, Nikkel A L, Bannon A W, Arneric S P, Decker M W (1998) Differences between the antinociceptive effects of the cholinergic channel activators A-85380 and (+/−)-epibatidine in rats. J Pharmacol Exp Ther 287(3):847-53.
Damaj M I, Fonck C, Marks M J, Deshpande P, Labarca C, Lester H A, Collins A C, Martin B R (2007) Genetic Approaches Identify Differential Roles for a4b2* Nicotinic Receptors in Acute Models of Antinociception in Mice. J Pharmacol Exp Ther 321:1161-9.
Damaj M I, Patrick G S, Creasy K R, Martin B R (1997) Pharmacology of lobeline, a nicotinic receptor ligand J Pharmacol Exp Ther 282(1):410-9.
Damaj M, Welch S P, Martin B R (1995) In vivo pharmacological effects of dihydro-β-erythroidine, a nicotinic antagonist, in mice. Psychopharmacology 117:67-73.
Decker M W, Rueter L E, Bitner R S (2004) Nicotinic acetylcholine receptor agonists: a potential new class of analgesics. Curr Top Med Chem 4(3):369-84.
Desrosiers G (2006) When opioid analgesia kills. Perspect Infirm 4(2):6-9.
Dineley-Miller K, Patrick J (1992) Gene transcripts for the nicotinic acetylcholine receptor subunit, beta4, are distributed in multiple areas of the rat central nervous system. Brain Res Mol Brain Res 16(3-4):339-44.
Dubuisson D, Dennis S G (1977) The formalin test: A quantitative study of the analgesic effects of morphine, meperidine, and brain stem stimulation in rats and cats. Pain 4:161-74.
Egleton R D, Brown K C, Dasgupta P (2008) Nicotinic acetylcholine receptors in cancer: multiple roles in proliferation and inhibition of apoptosis. Trends Pharmacol Sci. 29:151-8.
ElMallah M K, Pagliardini S, Turner S M, Cerreta A J, Falk D J, Byrne B J, Greer J J, Fuller D D (2015) Stimulation of Respiratory Motor Output and Ventilation in a Murine Model of Pompe Disease by Ampakines. Am J Respir Cell Mol Biol 53(3):326-35.
Funk G D, Greer J J (2013) The rhythmic, transverse medullary slice preparation in respiratory neurobiology: contributions and caveats. Respir Physiol Neurobiol 186(2):236-53.
Golder F J, Hewitt M W, McLeod J F (2013) Respiratory stimulant drugs in the post-operative setting. Resp Physiol Neurobiol 89:395-402.
Gotti C, Zoli M, Clementi F (2006) Brain nicotinic acetylcholine receptors: native subtypes and their relevance. Trends Pharmacol Sci 27: 482-91.
Gray P A, Janczewski W A, Mellen N, McCrimmon D R, Feldman J L (2001) Normal breathing requires preBötzinger complex neurokinin-1 receptor-expressing neurons. Nat Neurosci 4(9):927-30.
Gray P A, Rekling J C, Bocchiaro C M, Feldman J L (1999) Modulation of respiratory frequency by peptidergic input to rhythmogenic neurons in the preBötzinger complex. Science 286:1566-8.
Greer J J, Carter J, Al-Zubaidy Z A (1995) Opioid depression of respiration in neonatal rats. J Physiol 485:845-55.
Grupe M, Jensen A A, Ahring P K, Christensen J K, Grunnet M (2013) Unravelling the mechanism of action of NS9283, a positive allosteric modulator of (α4)3(β2)2 nicotinic ACh receptors. Br J Pharmacol 168(8):2000-10.
Guenther U, ManzkeT, Wrigge H, Dutschmann M, Zinserling J, Putensen C, Hoeft A (2009) The counteraction of opioid-induced ventilator depression by the serotonin1A-agonist 8-OH-DPAT does not antagonize antinociception in rats in situ and in vivo. Anesth Analg 108:1169-76.
Guenther U, Theuerkauf N U, Huse D, Boettcher M F, Wensing G, Putensen C, Hoeft A (2012) Selective 5-HT1A-R-agonist repinotan prevents remifentanil-induced ventilator depression and prolongs antinociception. Anesthesiology 116:56-64.
Guenther U, Wrigge H, Theuerkauf N, Boettcher M F, Wensing G, Zinserling J, Putensen C, Hoeft A (2010) Repinotan, a selective 5-HT1A-R-agonist, antagonizes morphine-induced ventilator depression in anesthetized rats. Anesth Analg 111:901-7.
Haji A, Okazaki M, Ohi Y, Yamazaki H, Takeda R (2003) Biphasic effects of morphine on bulbar respiratory neuronal activities in decerebrate cats. Neuropharmacology 45:368-79.
Hajiha M, DuBord M A, Liu H, Homer R L (2009) Opioid receptor mechanisms at the hypoglossal motor pool and effects on tongue muscle activity in vivo. J Physiol 587:2677-92.
Harvey S C, Maddox F N, Luetje C W (1996) Multiple determinants of dihydro-beta-erythroidine sensitivity on rat neuronal nicotinic receptor alpha subunits. J Neurochem 67(5):1953-9.
Hatori E, Sakuraba S, Kashiwagi M, Kuribayashi J, Tsujita M, Hosokawa Y, Takeda J, Kuwana S-I (2006) Association of nicotinic acetylcholine receptors with central respiratory control in isolated brainstem-spinal cord preparation of neonatal rats. Biol Res 39 (2):321-30.
Hussmann G P, Turner J R, Lomazzo E, Venkatesh R, Cousins V, Xiao Y, Yasuda R P, Wolfe B B, Perry D C, Rezvani A H, Levin E D, Blendy J A, Kellar K J (2012) Chronic sazetidine-A at behaviorally active doses does not increase nicotinic cholinergic receptors in rodent brain. J Pharmacol Exp Ther 343(2):441-50.
Iha H A, Kunisawa N, Shimizu S, Tokudome K, Mukai T, Kinboshi M, Ikeda A, Ito H, Serikawa T, Ohno Y (2017) Nicotine Elicits Convulsive Seizures by Activating Amygdalar Neurons. Front Pharmacol 8:57.
Jain K K (2004) Modulators of nicotinic acetylcholine receptors as analgesics. Curr Opin Investig Drugs 5(1):76-81.
Jarzyna D, Jungquist C R, Pasero C, Willens J S, Nisbet A, Oakes L, Dempsey S J, Santangelo D, Polomano R C (2011) American Society for Pain Management Nursing Guidelines on Monitoring for Opioid-Induced Sedation and Respiratory Depression. Pain management nursing 12(3): 118-45.
Jungquist C R, Willens J S, Dunwoody D R, Klingman K J, Polomano R C (2013) Monitoring for Opioid-Induced Advancing Sedation and Respiratory Depression: ASPMN Membership Survey of Current Practice. Pain Management Nursing 15(3):682-93.
Kaniakova M, Skrenkova K, Adamek S, Vyskocil F, Krusek J (2014) Different effects of lobeline on neuronal and muscle nicotinic receptors. Eur J Pharmacol 738:352-9.
Kessler E R, Shah M, Gruschkus S K, Raju A (2013) Cost and quality implications of opioid-based postsurgical pain control using administrative claims data from a large health system: Opioid-related adverse events and their impact on clinical and economic outcomes. Pharmacotherapy 33(4):383-91.
Kivell B M, Day D J, McDonald F J, Miller J H (2004) Developmental expression of mu and delta opioid receptors in the rat brainstem: evidence for a postnatal switch in mu isoform expression. Brain Res Dev Brain Res 148:185-96.
Kong H, Song J K, Yenugonda V M, Zhang L, Shuo T, Cheema A K, Kong Y, Du G H, Brown M L (2015) Preclinical studies of the potent and selective nicotinic α4β2 receptor ligand VMY-2-95. Mol Pharm 12(2):393-402.
Krystal A, Lippa A, Nasiek D, Krusinska E, Purcell R (2017) Opioids and sleep apnea: Antagonism of remifentanil-induced respiratory depression by CX1739 in two clinical models of opioid induced respiratory depression. Sleep 31st Annual Meeting of the Associated Professional Sleep Societies Vol 40:0571.
Kubin L, Fenik V (2004) Pontine cholinergic mechanisms and their impact on respiratory regulation. Respir Physiol Neurobiol 143(2-3):235-49.
Lalley P M (2006) Opiate slowing of feline respiratory rhythm and effects on putative medullary phase-regulating neurons. Am J Physiol Regul Integr Comp Physiol 290:R1387-96.
Launois S H, Pepin J L, Levy P (2007) Sleep apnea in the elderly: a specific entity? Sleep Med Rev 11(2):87-97.
Lee C H, Zhu C, Malysz J, Campbell T, Shaughnessy T, Honore P, Polakowski J, Gopalakrishnan M (2011) α4β2 neuronal nicotinic receptor positive allosteric modulation: an approach for improving the therapeutic index of α4β2 nAChR agonists in pain. Biochem Pharmacol 82(8):959-66.
Levin E D, Rezvani A H, Xiao Y, Slade S, Cauley M, Wells C, Hampton D, Petro A, Rose J E, Brown M L, et al. (2010) Sazetidine-A, a selective α4β2 nicotinic receptor desensitizing agent and partial agonist, reduces nicotine self-administration in rats. J Pharmacol Exp Ther 332:933-939.
Lippiello P M, Bencherif M, Gray J A, Peters S, Grigoryan G, Hodges H, Collins A C (1996) RJR-2403: a nicotinic agonist with CNS selectivity II. In vivo characterization. J Pharmacol Exp Ther 279(3): 1422-9.
Liu X, Sood S, Liu H, Homer R L (2005) Opposing muscarinic and nicotinic modulation of hypoglossal motor output to genioglossus muscle in rats in vivo. J Physiol 565:965-80.
Lorier A R, Funk G D, Greer J J (2010) Fentanyl induced suppression of hypoglossal motoneuron activity and ampakine therapy. PLoS ONE 5(1):e8766.
Lorier A R, Lipski J, Housley G D, Greer J J, Funk G D (2008) ATP sensitivity of preBötzinger complex neurones in neonatal rat in vitro: mechanism underlying a P2 receptor-mediated increase in inspiratory frequency. J Physiol 586(5):1429-46.
Lötsch J, Skarke C, Schneider, A Hummel T, Geisslinger G (2005) The 5-hydroxytryptamine 4 receptor agonist mosapride does not antagonize morphine-induced respiratory depression. Clin. Pharmacol. Ther 78:278-87.
Lynch G (2006) Glutamate-based therapeutic approaches: ampakines. Curr Opin Pharmacol 6:82-8.
Lynch G, Gall C M (2006) Ampakines and the threefold path to cognitive enhancement. Trends Neurosc 29(10):554-62.
Maurer J J, Sandager-Nielsen K, Schmidt H D (2017) Attenuation of nicotine taking and seeking in rats by the stoichiometry-selective α4β2 nicotinic acetylcholine receptor positive allosteric modulator NS9283. Psychopharmacology 234:475-84.
McLeod J F, Leempoels J M, Peng S X, Dax S L, Myers L J, Golder F J (2014) GAL-021, a new intravenous BKCa-channel blocker, is well tolerated and stimulates ventilation in healthy volunteers. Br J Anaesth. 113(5):875-83.
Menzaghi F, McClure D E, Lloyd G K (1998). Subtype-selective nAChR agonists for the treatment of neurological disorders: SIB-1508Y and SIB-1553A. In: Arneric S P, Brioni J D (eds). Neuronal
Miller D K, Lever J R, Rodvelt K R, Baskett J A, Will M J, Kracke G R (2007) Lobeline, a potential pharmacotherapy for drug addiction, binds to mu opioid receptors and diminishes the effects of opioid receptor agonists. Drug Alcohol Depend 89(2-3):282-91.
Montandon G, Qin W, Liu H, Ren J, Greer J J, Horner R L (2011) PreBotzinger complex neurokinin-1 receptor-expressing neurons mediate opioid-induced respiratory depression. J Neurosci 31(4):1292-301.
Moroni M, Zwart R, Sher E, Cassels B K, Bermudez I (2006) a4b2 nicotinic receptors with high and low acetylcholine sensitivity: pharmacology, stoichiometry, and sensitivity to long-term exposure to nicotine. Mol Pharmacol 70:755-768.
Oertel B G, Felden L, Tran P V, Bradshaw M, Angst M S, Schmidt H, Johnson S, Greer J J, Geisslinger G, Varney M A, Lotsch J (2010) Selective antagonism of opioid-induced respiratory depression by an AMPAKINE molecule in humans without loss of opioid analgesia. Clin Pharm Therap 87:204-11.
Oertel B G, Schneider A, Rohrbacher M, Schmidt H, Tegeder I, Geisslinger G, Lotsch J (2007) The partial 5-hydroxytryptamine1A receptor agonist buspirone does not antagonize morphine-induced respiratory depression in humans. Clin Pharmacol Ther 81:59-68.
Paleari L, Cesario A, Fini M, Russo P (2009) a7-Nicotinic receptor antagonists at the beginning of a clinical era for NSCLC and Mesothelioma? Drug Discov Today. 14:822-36.
Pandya A, Yakel J L (2011) Allosteric modulators of the α4β2 subtype of neuronal nicotinic acetylcholine receptors. Biochem Pharmacol. 82(8):952-8.
Ren J, Ding X, Funk G D, Greer J J (2009) Ampakine CX717 protects against fentanyl-induced respiratory depression and lethal apnea in rats. Anesthesiology 110(6):1364-70.
Ren J, Ding, X, Greer J J (2015) 5-HT1A receptor agonist befiradol reduces fentanyl-induced respiratory depression, analgesia, and sedation in rats. Anesthesiology 122:424-34.
Ren J, Ding X, Greer J J (2012) Respiratory depression in rats induced by alcohol and barbiturate and rescue by ampakine CX717. J Appl Physiol (1985) 113(7):1004-11.
Ren J, Lenal F, Yang M, Ding X, Greer J J (2013) Coadministration of the AMPAKINE CX717 with propofol reduces respiratory depression and fatal apneas. Anesthesiology 118(6):1437-45.
Ren J, Poon B Y, Tang Y, Funk G D, Greer J J (2006) Ampakines alleviate respiratory depression in rats. Am J Resp Crit Care Med 174:1384-91.
Rode F, Munro G, Hoist D, Nielsen E O, Troelsen K B, Timmermann D B, Rønn L C, Grunnet M (2012) Positive allosteric modulation of α4β2 nAChR agonist induced behaviour. Brain Res 1458:67-75.
Roozekrans M, Olofsen E, van der Schrier R, van Gerven J, Peng S, McLeod J, Dahan A: Reversal of opioid-induced respiratory depression by BK-channel blocker GAL021: A pharmacokinetic-pharmacodynamic modeling study in healthy volunteers. Clin Pharmacol Ther 2015; 97(6):641-49
Ruangkittisakul A, Schwarzacher S W, Secchia L, Poon B Y, Ma Y, Funk G D, Ballanyi K (2006) High sensitivity to neuromodulator-activated signaling pathways at physiological [K+] of confocally imaged respiratory center neurons in on-line-calibrated newborn rat brainstem slices. J Neurosci 26: 11870-80.
Rueter L E, Donnelly-Roberts D L, Curzon P, Briggs C A, Anderson D J, Bitner R S (2006) A-85380: a pharmacological probe for the preclinical and clinical investigation of the a4b2 nicotinic acetylcholine receptor. CNS Drug Rev 12(2):100-12.
Rueter L E, Kohlhaas K L, Curzon P, Surowy C S, Meyer M D (2003) Peripheral and central sites of action for A-85380 in the spinal nerve ligation model of neuropathic pain. Pain 103:269-76.
Rueter L E, Meyer M D, Decker M W (2000) Spinal mechanisms underlying A-85380-induced effects on acute thermal pain. Brain Res 872:93-101.
Sahibzada N, Ferreira M, Wasserman A M, Taveira-DaSilva A M, Gillis R A (2000) Reversal of morphine-induced apnea in the anesthetized rat by drugs that activate 5-hydroxytryptamine(1A) receptors. J Pharmacol Exp Ther 292:704-13.
Semenova S, Contet C, Roberts A J, Markou A (2012) Mice lacking the β4 subunit of the nicotinic acetylcholine receptor show memory deficits, altered anxiety- and depression-like behavior, and diminished nicotine-induced analgesia. Nicotine Tob Res 14(11):1346-55.
Shao X M, Feldman J L (2002) Pharmacology of nicotinic receptors in preBötzinger complex that mediate modulation of respiratory pattern. J Neurophysiol 88: 1851-8.
Shook J E, Watkins W D, Camporesi E M (1990) Differential roles of opioid receptors in respiration, respiratory disease, and opiate-induced respiratory depression. Am Rev Respir Dis 142(4):895-909.
Sloan J W, Martin W R, Bostwick M, Hook R, Wala E (1988) The comparative binding characteristics of nicotinic ligands and their pharmacology. Pharmacol Biochem Behav 30(1):255-67.
Smith J C, Ellenberger H H, Ballanyi K, Richter D W, Feldman J L (1991) Pre-Botzinger complex: A brainstem region that may generate respiratory rhythm in mammals. Science 254:726-9.
Smith J C, Greer J J, Liu G S, Feldman J L (1990) Neural mechanisms generating respiratory pattern in mammalian brain stem-spinal cord in vitro. I. Spatiotemporal patterns of motor and medullary neuron activity. J Neurophysiol 64:1149-69.
Sullivan J P, Donnelly-Roberts D, Briggs C A, et al (1996) A-85380 [3-(2(s)-azetidinylmethoxy) pyridine]: In vitro pharmacological properties of a novel, high affinity a4b2 nicotinic acetylcholine receptor ligand. Neuropharmacology 35:725-34.
Swarm R A, Karanikolas M, Kalauokalani D (2001) Pain treatment in the perioperative period. Curr Probl Surg 38(11):835-920.
Takeda S, Eriksson L I, Yamamoto Y, Joensen H, Onimaru H, Lindahl S G (2001) Opioid action on respiratory neuron activity of the isolated respiratory network in newborn rats. Anesthesiology 95(3):740-9.
Timmermann D B, Sandager-Nielsen K, Dyhring T, Smith M, Jacobsen A-M, Nielsen EØ, Grunnet M, Christensen J K, Peters D, Kohlhaas K, Olsen G M, Ahring P K (2012) Augmentation of cognitive function by NS9283, a stoichiometry-dependent positive allosteric modulator of α2- and α4-containing nicotinic acetylcholine receptors. Br J Pharmacol 167(1):164-82.
Tournier J M, Birembaut P (2011) Nicotinic acetylcholine receptors and predisposition to lung cancer. Curr Opin Oncol. 23:83-7.
Turner S M, ElMallah M K, Hoyt A K, Greer J J, Fuller D D (2016) Ampakine CX717 potentiates intermittent hypoxia-induced hypoglossal long-term facilitation. J Neurophysiol 16(3):1232-8
Vernier J M, El-Abdellaoui H, Holsenback H, Cosford N D, Bleicher L, Barker G, Bontempi B, Chavez-Noriega L, Menzaghi F, Rao T S, et al. (1999) 4-{[2-(1-Methyl-2-pyrrolidinyl)ethyl]thio}phenol hydrochloride (SIB-1553A): a novel cognitive enhancer with selectivity for neuronal nicotinic acetylcholine receptors. J Med Chem 42:1684-1686.
Wada E, Wada K, Boulter J, Deneris E, Heinemann S, Patrick J, Swanson L W (1989) Distribution of a2, a3, a4, and b2 neuronal nicotinic receptor subunit mRNAs in the central nervous system: a hybridization histochemical study in the rat. J Comp Neurol 284:314-35.
Xiao Y, Fan H, Musachio J L, Wei Z L, Chellappan S K, Kozikowski A P, Kellar K J (2006) Sazetidine-A, a novel ligand that desensitizes a4b2 nicotinic acetylcholine receptors without activating them. Mol Pharmacol 70:1454-1460.
Yenugonda V M, Xiao Y, Levin E D, Rezvani A H, Tran T, Al-Muhtasib N, Sahibzada N, Xie T, Wells C, Slade S, Johnson J E, Dakshanamurthy S, Kong H S, Tomita Y, Liu Y, Paige M, Kellar K J, Brown M L (2013) Design, synthesis and discovery of picomolar selective α4β2 nicotinic acetylcholine receptor ligands. J Med Chem 56(21):8404-21.
Zaninetti M, Tribollet E, Bertrand D, Raggenbass M (1999) Presence of functional neuronal nicotinic acetylcholine receptors in brainstem motoneurons of the rat. Eur J Neurosci 11:2737-48.
Zhu C Z, Chin C L, Rustay N R, Zhong C, Mikusa J, Chandran P, Salyers A, Gomez E, Simler G, Lewis L G, Gauvin D, Baker S, Pai M, Tovcimak A, Brown J, Komater V, Fox G B, Decker M W, Jacobson P B, Gopalakrishnan M, Lee C H, Honore P (2011) Potentiation of analgesic efficacy but not side effects: co-administration of an α4β2 neuronal nicotinic acetylcholine receptor agonist and its positive allosteric modulator in experimental models of pain in rats. Biochem Pharmacol. 82(8):967-76.
Zimmer M B, Goshgarian H G (2005) Spontaneous crossed phrenic activity in the neonatal respiratory network. Exp Neurol 194(2):530-40.
Zwart R, Carbone A L, Moroni M, Bermudez I, Mogg A J, Folly E A, Broad L M, Williams A C, Zhang D, Ding C, Heinz B A, Sher E (2008) Sazetidine-A is a potent and selective agonist at native and recombinant alpha 4 beta 2 nicotinic acetylcholine receptors. Mol Pharmacol 73(6):1838-43.

All publications mentioned in this paragraph are provided solely for their disclosure prior to the filing date of the present application and are not incorporated herein by reference.
Marubio L M, del Mar Arroyo-Jimenez M, Cordero-Erausquin M, Léna C, Le Novère N, de Kerchove d'Exaerde A, Huchet M, Damaj M I, Changeux J P (1999) Reduced antinociception in mice lacking neuronal nicotinic receptor subunits. Nature 398:805-10.
Olsen J A, Kastrup J S, Peters D, Gajhede M, Balle T, Ahring P K (2013) Two distinct allosteric binding sites at α4β2 nicotinic acetylcholine receptors revealed by NS206 and NS9283 give unique insights to binding activity-associated linkage at cys-loop receptors. J Bio Chem 288 (50): 35997-6006.
Ren J, Lee S, Pagliardini S, Gerard M, Stewart C L, Greer J J, Wevrick R (2003) Absence of Ndn, encoding the Prader-Willi syndrome-deleted gene necdin, results in congenital deficiency of central respiratory drive in neonatal mice. J Neurosci 23(5):1569-73.
Wu J, Liu Q, Yu K, Hu J, Kuo Y P, Segerberg M, St John P A, Lukas R J (2006) Roles of nicotinic acetylcholine receptor beta subunits in function of human alpha4-containing nicotinic receptors. J Physiol 576(Pt 1):103-18.

What is claimed is:

1. A method of treating, preventing, or ameliorating respiratory depression and overdose induced by an opioid, or other cause of respiratory depression in a subject, comprising administering to the subject an effective amount of a compound capable of activating a neuronal heteromeric nicotinic acetylcholine receptor or a composition comprising same.

2. The method of claim 1, wherein the other cause of respiratory depression is selected from a non-opioid drug, obstructive sleep apnea, central sleep apnea, apnea of prematurity, hypoxia, Prader-Willi Syndrome, Rett Syndrome, Pompe Disease, Cheyne-Stokes breathing, neuronal degeneration, stroke, heart failure, brain trauma, Parkinson's Disease, or spinal cord injury.

3. The method of claim 1, wherein the compound is selected from a positive allosteric modulator of the neuronal heteromeric nicotinic acetylcholine receptor or a nicotinic acetylcholine agonist selected from a full agonist or a partial agonist.

4. The method of claim 3, wherein the neuronal heteromeric nicotinic acetylcholine receptor is an α4β2 nicotinic acetylcholine receptor.

5. The method of claim 3, wherein the neuronal heteromeric nicotinic acetylcholine receptor is an α4β4 nicotinic acetylcholine receptor.

6. The method of claim 3, wherein the neuronal heteromeric nicotinic acetylcholine receptor is a β4 containing nicotinic acetylcholine receptor.

7. The method of claim 3, wherein the neuronal heteromeric nicotinic acetylcholine receptor is an α3β4 nicotinic acetylcholine receptor.

8. The method of claim 1, wherein the compounds is selected from the group consisting of 3-(2(s)-azetidinylmethoxy) pyridine (A85380), 2-((2R,6S)-6-((S)-2-Hydroxy-2-phenylethyl)-1-methylpiperidin-2-yl)-1-phenylethanone (lobeline), (E)-N-Methyl-4-(3-pyridinyl)-3-butene-1-amine (Rivanicline), 6-[5-[(2S)-2-Azetidinylmethoxy]-3-pyridinyl]-5-hexyn-1-ol (sazetidine-A), 3-[(2S)-2-Azetidinylmethoxy]-5-(2-phenylethynyl)-pyridine (VMY-2-95), (±)-4-[2-((N-methyl)-2-pyrrolidinyl)ethyl]thiophenol (SIB-1533A), 3-[3-(3-Pyridinyl)-1,2,4-oxadiazol-5yl]benzonitrile (NS9283), 5-{[(2R)-Azetidin-2-yl]methoxy}-2-chloropyridine (Tebanicline, Ebanicline, ABT-594), (1S,5S)-3-(5,6-Dichloro-3-pyridinyl)-3,6-diazabicyclo[3.2.0]heptane (Sofinicline, ABT-894, A-422894), 2-Methyl-3-{[(2S)-pyrrolidin-2-yl]methoxy}pyridine (Pozanicline, ABT-089), 3-methyl-5-[(2S)-1-methylpyrrolidin-2-yl]-1,2-oxazole (ABT 418), and 7,8,9,10-Tetrahydro-6,10-methano-6H-pyrazino[2,3-h] [3]benzazepine (Varenicline, Chantix, Champix).

9. The method of claim 4, wherein the compound is a full agonist comprising 3-(2(s)-azetidinylmethoxy) pyridine (A85380).

10. The method of claim 4, wherein the compound is a partial agonist comprising 2-((2R,6S)-6-((S)-2-Hydroxy-2-phenylethyl)-1-methylpiperidin-2-yl)-1-phenylethanone (lobeline).

11. The method of claim 4, wherein the compound is a partial agonist comprising (E)-N-Methyl-4-(3-pyridinyl)-3-butene-1-amine or (E)-metanicotine (Rivanicline).

12. The method of claim 4, wherein the compound is a partial agonist comprising 6-[5-[(2S)-2-Azetidinylmethoxy]-3-pyridinyl]-5-hexyn-1-ol (sazetidine-A).

13. The method of claim 4, wherein the compound is a partial agonist comprising 3-[(2S)-2-Azetidinylmethoxy]-5-(2-phenylethynyl)-pyridine (VMY-2-95).

14. The method of claim 4, wherein the compound is a partial agonist comprising (±)-4-[2-((N-methyl)-2-pyrrolidinyl)ethyl]thiophenol (SIB-1533A).

15. The method of claim 4, wherein the compound is a positive allosteric modulator comprising 3-[3-(3-Pyridinyl)-1,2,4-oxadiazol-5yl]benzonitrile (NS9283).

16. The method of claim 5, wherein the compound is a full agonist comprising (±)-4-[2-((N-methyl)-2-pyrrolidinyl)ethyl]thiophenol (SIB-1533A).

17. The method of claim 6, wherein the compound is a full agonist comprising 2-((2R,6S)-6-((S)-2-Hydroxy-2-phenylethyl)-1-methylpiperidin-2-yl)-1-phenylethanone (lobeline).

18. The method of claim 6, wherein the compound is a full agonist comprising (±)-4-[2-((N-methyl)-2-pyrrolidinyl)ethyl]thiophenol (SIB-1533A).

19. The method of claim 7, wherein the compound is a partial agonist comprising 2-((2R,6S)-6-((S)-2-Hydroxy-2-phenylethyl)-1-methylpiperidin-2-yl)-1-phenylethanone (lobeline).

20. The method of claim 7, wherein the compound is a full agonist comprising (±)-4-[2-((N-methyl)-2-pyrrolidinyl)ethyl]thiophenol (SIB-1533A).

21. The method of claim 1, wherein the respiratory depression and the overdose is treated, prevented, or ameliorated with intravenous or intramuscular administration of the compound.

22. The method of claim 1, wherein the opioid comprises fentanyl.

23. Use of an effective amount of a compound capable of activating a neuronal heteromeric nicotinic acetylcholine receptor or a composition comprising same to treat, prevent, or ameliorate respiratory depression and overdose induced by an opioid, or other cause of respiratory depression in a subject.

24. A method of inducing analgesia, anesthesia, or sedation in a subject, while simultaneously treating, preventing, or ameliorating respiratory depression and overdose induced by an opioid, or other cause of respiratory depression, comprising administering to the subject an effective amount of a compound capable of activating a neuronal heteromeric nicotinic acetylcholine receptor or a composition comprising same.

25. Use of an effective amount of a compound capable of activating a neuronal heteromeric nicotinic acetylcholine receptor or a composition comprising same to induce analgesia, anesthesia, or sedation in a subject, while simultaneously treating, preventing, or ameliorating respiratory depression and overdose induced by an opioid, or other cause of respiratory depression.

26. The method as claimed in claim 1, wherein the compound is 3-(2(s)-azetidinylmethoxy) pyridine (A85380) to alleviate respiratory depression caused by weak endogenous excitatory drive.

27. The method as claimed in claim 1, wherein the compound is 3-(2(s)-azetidinylmethoxy) pyridine (A85380) to alleviate respiratory depression caused by spinal cord injury.

28. The method of claim 3, wherein the neuronal heteromeric nicotinic acetylcholine receptor is an α4β2 nicotinic acetylcholine receptor and a α6β2 nicotinic acetylcholine receptor.
